(19) United States
(12) Patent Application Publication
(10) Pub. No.: US 2025/0099465 A1
(43) Pub. Date: Mar. 27, 2025

(54) REMIBRUTINIB FOR USE IN THE TREATMENT OF HIDRADENITIS SUPPURATIVA

(71) Applicant: NOVARTIS AG, Basel (CH)

(72) Inventors: Souvik BHATTACHARYA, Weymouth, MA (US); Bruno BIETH, Saint-Louis (FR); Bruno CENNI, Sissach (CH); Gordon GRAHAM, Huningue (FR); Michael JUHNKE, Basel (CH); Christian LOESCHE, Hegenheim (FR); Karin RAPP, Lörrach (DE); Kim-Hien SIN, Saint-Louis (FR); Jonas Benjamin ZIERER, Basel (CH)

(21) Appl. No.: 18/729,542

(22) PCT Filed: Feb. 27, 2023
(86) PCT No.: PCT/IB2023/051787

§ 371 Date: Jul. 17, 2024

Related U.S. Application Data
(60) Provisional application No. 63/314,572, filed on Feb. 28, 2022.
(60) Provisional application No. 63/369,016, filed on Jul. 21, 2022.

(51) Int. Cl. A61K 31/505; A61K 45/06; A61P 17/00

(52) U.S. Cl. CPC A61K 31/505; A61K 45/06; A61P 17/00

(57) ABSTRACT

The present disclosure relates to methods for treating and/or preventing Hidradenitis Suppurativa in a subject having such disease or condition comprising administering a therapeutically effective dose of LOU064. Also disclosed are medicaments, dosing regimens, pharmaceutical compositions, combinations, dosage forms, and kits for use in the disclosed uses and methods.

### TECHNICAL FIELD

The present invention concerns LOU064 or a pharmaceutically acceptable salt thereof for use in the effective and safe treatment of Hidradenitis suppurativa (HS).

### BACKGROUND OF THE INVENTION

Hidradenitis suppurativa (HS) (also referred to as acne inversa or Verneuil's disease) is a chronic, recurring, inflammatory disease characterized by deep-seated nodules, sinus tracts, and abscesses that lead to fibrosis in the axillary, inguinal, breast-fold, and anogenital regions. (Revuz and Jemec (2016) Dermatol Clin 34:1-5; Jemec G B. (2012) N Engl J Med 366:158-64). It is associated with substantial pain and comorbidities, including metabolic, psychiatric, and autoimmune disorders, as well as an increased risk of skin cancer. (Revuz (2016); Shlyankevich et al. (2014) J Am Acad Dermatol 71:1144-50; Kohorst et al (2015) J Am Acad Dermatol 73:S27-35; Wolkenstein et al. (2007) J Am Acad Dermatol 56:621-3). The average HS prevalence rates amongst different races/ethnicities were highest in African American populations (1.3%), lowest in Hispanics/Latinos (0.07%), and intermediate amongst Caucasian populations (0.75%) (Sachdeva et al. (2021) J. Cutan Med Surg.: 25(2):177-187.

Reported prevalence rates of HS vary from <1% to 4% of the population. [Shlyankevich et al. (2014); Cosmatos et al. (2013) J Am Acad Dermatol 68:412-9; Davis et al. (2015) Skin Appendage Disord 1:65-73; Revuz et al. (2008) J Am Acad Dermatol 59:596-601; McMillan K. (2014) Am J Epidemiol 179:1477-83; Garg et al. (2017) J Am Acad Dermatol, 77(1):118-122; Jemec et al. (1996) J Am Acad Dermatol 35:191-4]. However, the true prevalence is difficult to ascertain because HS is underdiagnosed, and estimates fluctuate with study design, population, and geographic location. [Miller et al. (2016) Dermatol Clin 34:7-16]. Although the National Institutes of Health (NIH) does not classify HS as a rare disease, experts generally consider the prevalence of the disease to be <1% of the United States (US) population. [Cosmatos et al. (2013); Genetic and Rare Diseases Information Center. National Institutes of Health. Hidradenitis suppurativa. Available at: //rarediseases.info.nih.gov/diseases/6658/hidradenitis-suppurativa. Accessed Mar. 20, 2017; Gulliver et al. (2016) Rev Endocr Metab Disord 17:343-51; Garg et al. 2007].

Current treatment for HS consists of topical and/or systemic antibiotics, hormonal interventions, retinoids, and, in selected cases, immunosuppressants, biologics such as the tumor necrosis factor [TNF] inhibitor monoclonal antibody adalimumab, the only approved drug in HS and often as last resort large surgical excision. [Gulliver et al. (2016); Zouboulis et al. (2015) J Eur Acad Dermatol Venereol 29:619-4414-16; Kimball et al. (2016) N Engl J Med 375:422-34]. As the lesions are painful, the patients need very often analgesics and pain relieve on top of anti-inflammatory treatments.

However, symptom control and lesion resolution are inconsistent among treatments and randomized controlled clinical trial results providing evidence-based data are lacking for most treatments and only adalimumab is approved. While antibiotic therapy is used for long-term treatment for months and even years, antibiotic therapy may thus results in resurgence of antibacterial resistance. Oral treatment with retinoids poses teratogenicity concerns in a population that is sexually active and mostly female. Moreover, the effectiveness of inflammatory drugs, such as dapsone, fumarates and cyclosporine, is based on small case studies with varying results and these molecules are not systematically used. Because of these inconsistent outcomes, and the severity of the HS disease, HS patients utilize healthcare in high-cost settings (e.g., emergency department and inpatient care) more frequently than patients with other chronic inflammatory skin conditions. (Khalsa et al. (2016) J Am Acad Dermatol 73:609-14; Kirby et al. (2014) JAMA Dermatol 150:937-44).

The immunopathogenesis of HS is poorly understood, with widely and somewhat discordant pathogenic mechanisms proposed. Complement activation has been implicated in the pathogenesis of HS through elevated concentration of the complement fragment C5a in skin and blood of patients with HS (Kanni T. et al. Br J Dermatol. 2018; 179(2):413-419). C5a is a major chemoattractant for neutrophils, a cell population that is prominent in actively inflamed HS skin (Lima A L. et al. Br J Dermatol. 2016; 174(3):514-520). Furthermore, increased levels of neutrophils releasing neutrophil extracellular traps (NETs) in a process called NETosis have been demonstrated in both blood and skin of patients with HS (Byrd A S et al., Sci transl Med. 2019: 11(508).eaav5908), along with increased type I IFN responses and activation of plasmacytoid dendritic cells (pDCs). Other studies have suggested involvement of the proinflammatory cytokines IL-17A and IFN-γ (Hotz C. et al.; J. Invest Dermatol., 2016:136(9):1768-1780; Vossen ARJV et al. Allergy. 2019, 74(3):631-634), as well as keratinocyte-derived IL-6 and IL-1p. Additional transcriptomic studies have noted the increased expression of immunoglobulins and plasma cell signatures in HS (Hoffman L K. Et al. PLoS One. 2018; 13(9):e0203672), but a clear and comprehensive view of the immunopathogenic mechanisms involved in HS has been lacking.

Gudjonsson et al. characterized the inflammatory responses in HS in depth using proteomic and transcriptomic approaches and approaches such as single cell RNA sequencing or scRNAseq. Such studies revealed immune responses centered on IFN-γ, IL-36, and TNF, with lesser contribution from IL-17A. Gudjonsson et al. further identified B cells and plasma cells, with associated increases in immunoglobulin production and complement activation, as players in HS pathogenesis, with Bruton's tyrosine kinase (BTK) and spleen tyrosine kinase (SYK) pathway activation as a central signal transduction network in HS (JCI Insight. 2020; 5(19):e139930. https://doi.org/10.1172/jci.insight.139930).

The role for B cells in HS pathogenesis has also been suggested in previous reports (Van der Zee et al. 2012, 166:98-106; Musilova et al. J Invest Dermatol 2020, 140:1091-1094). Furthermore, the role for B cells in HS pathogenesis is also highlighted by two recent studies, one demonstrating anti-inflammatory effects of rituximab in HS explant cultures (Vossen et al., 2019, 181:314-23) and the other a case study showing successful treatment of HS using B-cell depletion with rituximab (Takahashi et al., 2018, 45:e116-7). But further research is warranted to determine if targeting B cells may be an effective treatment.

BTK is an essential kinase for signaling through FcεRI in human mast cells and basophils. Because it is also crucial for B cell maturation, BTK has been pharmacologically targeted for the treatment of B cell malignancies. There are now three FDA-approved BTK inhibitors (BTKis) in the USA. Ibrutinib (brand name Imbruvica®; Pharmacyclics, and AbbVie), acalabrutinib (Calquence®; Acerta and AstraZeneca), and zanubrutinib (Brukinsa®; BeiGene) are all oral, covalent small molecule inhibitors of BTK.

Furthermore, many BTKis in development are in clinical trials for the treatment of auto-immune diseases such as rheumatoid arthritis, multiple sclerosis, and systemic lupus erythematosus. For example, Acalabrutinib and Zanubrutinib as well as the novel compounds ONO-4059 (Tirabrutinib), HM71224 (Poseltinib) and ABBV-105 (Upadacitinib) are currently being tested for their efficacy in B cell malignancies and/or autoimmune diseases such as rheumatoid arthritis (RA), Sjögren's Syndrome (SjS) and systemic lupus erythematosus (SLE). Additionally, evobrutinib, tolebrutinib and fenebrutinib have entered phase Ill studies in multiple sclerosis (MS) patients, orelabrutinib is tested in a phase II study and BIIB091 was tested in a phase I study for efficacy in the treatment of MS.

Despite suggestion that BTK inhibitors may be useful for the treatment of HS, there is no existing animal models of this disease and BTK inhibitors have not yet shown to treat HS in human. Furthermore, due to the lack of selectivity of some earlier developed BTK inhibitors (e.g. acalabrutinib, ibrutinib), those BTK inhibitors may not be suitable for non-malignant indications, especially for the treatment of indications which requires long term/chronic and safe use, and/or in a pediatric or adolescent population. The most common side effects of the currently approved BTK inhibitors include nausea, diarrhea, rash, infection, cytopenias, bleeding and cardiac arrhythmias. Particularly, the long-term toxicity profile of ibrutinib, a first-in-class inhibitor, is well characterized and includes a clinically significant incidence of cardiac arrhythmias, bleeding, infection, diarrhea, arthralgias, and hypertension. Acalabrutinib, the initial second-generation BTKi to earn approval from the US Food and Drug Administration, demonstrates improved kinase selectivity for BTK, with commonly observed adverse reactions including infection, headache, and diarrhea (Hematology Am Soc Hematol Educ Program. 2020 Dec. 4; 2020(1): 336-345)

Long-term safety data is not available for BTK inhibitor currently in development but available safety data for fenebrutinib may suggest that the biggest obstacle that BTK inhibitors may face is whether side effects during chronic use will be acceptable. For example, a major event noted for fenebrutinib's phase IIa trial was transient grade 3 elevations of ALT and/or AST in 8.3% and 6.3% of the subjects in the higher dose treatment arms. Some of the side effects already observed for some BTKis may limit their use for treating or preventing non-malignant indications, especially if would require a long-term administration. Additionally none of the FDA-approved BTK inhibitors have been approved in children and adolescents. Therefore, until safety data in children is available it is unknown whether BTK inhibitors will be a viable option HS remains difficult to treat, with only one biologic therapy, the anti-TNF agent adalimumab, currently approved for its treatment, and even with adalimumab therapeutic responses are suboptimal in nearly 40% of patients. The disease is physically and psychologically debilitating, there is a clear unmet need to provide safe and effective long-term treatments for HS patients, in particular oral treatments.

### BRIEF DESCRIPTION OF THE FIGURES

FIG. 1: transcriptomics signature enrichment (gene over expressed in lesional HS samples)

FIG. 2: LOU signature (LOU064 suppressed genes) in the (bulk) transcriptomic dataset of hidradenitis suppurativa

FIG. 3: Simulation of Spleen BTK occupancy at steady state.

FIG. 4: (A) Trough over 24 hours of BTK Occupancy at steady state. Graph showing the median prediction as point and the vertical lines showing the 95% prediction interval. (B) Average over 24 hours of BTK Occupancy at steady state. Graph showing the median prediction as point and the vertical lines showing the 95% prediction interval.

FIG. 5: Preferred particle size distribution of nanosized LOU064.

### SUMMARY OF THE INVENTION

The problem underlying the present invention is to provide a safe and efficacious treatment or prevention of HS. In particular, it is an object of the present invention to provide a safe and efficacious long-term treatment or prevention of HS.

It is also an object of the invention to provide an improved treatment and/or prevention of HS. For example, it is an object of the invention to provide a treatment or prevention of HS which is more effective than anti-TNF therapy, e.g. adalimumab.

Accordingly, disclosed herein are methods of preventing or treating HS, comprising administering a therapeutically effective dose of a BTK inhibitor to a subject in need thereof. Particularly useful for the method of the invention is a BTK inhibitor (reversible or irreversible) which is selective against other structurally similar Tec family kinases such as BMX, ITK and TXK. More particularly useful for the invention is a BTK inhibitor which is selective for BTK over Tec (e.g. a BTK/tec selectivity of at least 10 fold, at least 20 fold, at least 30 fold)

Also disclosed herein are methods of preventing or treating HS, comprising administering a therapeutically effective dose of LOU064 to a subject in need thereof.

Furthermore, the invention further provides a method of treating or preventing HS in a subject in need thereof, comprising administering to said subject a therapeutically effective dose of LOU064 or a pharmaceutically acceptable salt thereof with one or more therapeutic agents.

In another aspect of the invention, the present invention provides a BTK inhibitor for use in the treatment and/or prevention of HS, in a patient in need of such treatment and/or prevention.

In another aspect of the invention, the present invention provides LOU064 for use in the treatment and/or prevention of HS, in a patient in need of such treatment and/or prevention.

The invention further relates to combinations of LOU064 with one or more additional therapeutic agents, for use in the treatment or prevention of HS, in a patient in need of such treatment and/or prevention.

A further subject of the present invention is a method for the manufacture of a medicament for use in the treatments described above.

Further aspects, advantageous features and preferred embodiments of the present invention which are summarized in the following embodiments E1-E32, respectively alone or in combination, contribute to solving the object of the invention:
E1. A method of treating or preventing hidradenitis suppurativa (HS), comprising administering to a subject in need thereof a therapeutically effective dose of a BTK inhibitor, e.g. a selective BTK inhibitor, e.g. LOU064.
E2. The method according to E1, wherein the therapeutically effective dose of LOU064 is from about 50 mg to about 200 mg daily.
E3. The method according to E2, wherein the therapeutically effective dose of LOU064 is from about 25 mg twice daily to about 100 mg twice daily.
E4. The method according to E2, wherein the therapeutically effective dose of LOU064 is about 25 mg twice daily.
E5. The method according to E2, wherein the therapeutically effective dose of LOU064 is about 100 mg twice daily.
E6. The method according to any one of the preceding embodiments, wherein LOU064 is administered for a short term, e.g. less than 6 months, preferably less than 3 months.
E7. The method according to E6, wherein LOU064 is administered during up to 16 weeks, e.g. 4, 12, or 16 weeks.
E8. The method according to any one of embodiments E1 to E5, wherein LOU064 is administered for a long term, e.g. more than 6 months, preferably more than a year, chronic use.
E9. The method according to any one of the preceding embodiments, wherein LOU064 is administered as a monotherapy.
E10. The method according to any one of the preceding embodiments, wherein LOU064 is not administered concomitantly with a strong inhibitor of CYP3A.
E11. The method according to any one of the preceding embodiments, wherein LOU064 is not administered concomitantly with a strong inhibitor of CYP3A4.
E12. The method according to any one of embodiments E1 to E8, wherein LOU064 is administered in a combination therapy with one or more therapeutic agents.
E13. The method according to E12, wherein the patient is additionally treated with at least one topical medication and at least one antiseptic in combination with LOU064.
E14. The method according to any one of embodiments E1 to E11, wherein, prior to treatment with LOU064, the patient has not been previously treated with a systemic agent or a topical treatment for HS.
E15. The method according to any of the above embodiments, wherein the patient is selected according to at least one of the following criteria:
a) the patient has moderate to severe HS;
b) prior to treatment with the LOU064, the patient has at least 3 inflammatory lesions; or
c) prior to treatment with the LOU064, the patient does not have extensive scarring no more than 15 fistulae) as a result of HS;
d) the patient has a clinical diagnosis of HS for at least 12 months;
e) the patient has at least two anatomical areas involved with HS lesions.
E16. The method according to any of the above embodiments, wherein said patient achieves by week 16 of treatment at least one of the following:
a) a simplified HiSCR;
b) a HiSCR clinical result (HiSCR50, HiSCR75 or HiSCR90)
c) a reduction in HS flares:
d) a skin pain reduction as measured by pain NRS; e.g. pain NRS30
e) a itch reduction as measured by itch NRS; e.g. itch NRS30
f) a reduction in total or different types of HS inflammatory lesion counts; e.g. AN50, AN75, AN90 or AN100;
g) a reduction in IHS4 score;
h) a reduction of ≤6 as measured by the DLQI; and/or
i) an improvement in DLQI.
E17. The method according to any of the above embodiments, wherein, when said method is used to treat a population of patients with moderate to severe HS, at least 40% of said patients achieve by week 16 of treatment at least one of the following:
a) a simplified HiSCR;
b) a HiSCR50;
c) a HiSCR75
d) a HiSCR90.
E18. The method according to any of embodiments E1 to E16, wherein, when said method is used to treat a population of patients with moderate to severe HS, at least 25% of said patients achieve an NRS30 response (e.g. a pain NRS30 or an itch NRS30 response) by week 16 of treatment.
E19. The method according to any of the embodiments E1 to E16, wherein, when said method is used to treat a population of patients with moderate to severe HS, less than 15% of said patients experience an HS flare during 16 weeks of treatment.
E20. The method according to any of the embodiments E1 to E16, wherein the patient has at least one of the followings as early as one or two weeks after the first dose of LOU064:
a) a reduction in pain, as measured by pain NRS, (e.g. pain NRS30)
b) a reduction in itch, as measured by itch NRS, (e.g. skin itch NRS30)
b) a reduction in CRP, as measured using a standard CRP assay, e.g. at least 25%.
E21. The method according to any of the above embodiments, wherein said patient achieves a sustained response 3 months after the end of treatment, as measured by inflammatory lesion count, e.g. AN50, Hidradenitis Suppurativa Clinical Response (HiSCR) (e.g. sHiSCR, HiSCR50, HiSCR75 or HiSCR90), itch or pain Numerical Rating Scale (NRS), Hidradenitis Suppurativa-Physician Global Assessment (HS-PGA), severity assessment of HS (SASH), international HS severity score system (IHS4) or Dermatology Life Quality Index (DLQI).
E22. The method according to E21, wherein said patient achieves a sustained response 3 months after the end of treatment, as measured by the simplified HiSCR (sHiSCR), HiSCR50, HiSCR75 or HiSCR90.
E23. The method according to any of the above embodiments, wherein the LOU064 is disposed in a pharmaceutical composition, wherein said pharmaceutical composition comprises one or more pharmaceutically acceptable carriers, each of which is independently selected from a filler, a lubricant, a binder, a desintegrant and a glidant.
E24. The method according to E23, wherein the pharmaceutical composition is in tablet or capsule form.
E25. The method according to embodiment E23 or E24, wherein the pharmaceutical composition comprises nanosized particles of LOU064.
E26. The method according to E25, wherein the pharmaceutical composition comprises nanosized particles of LOU064 having a mean particle size as measured by PCS of between about 50 nm to about 750 nm.
E27. The method according to any one of embodiments E23 to E26, wherein the pharmaceutical composition comprises LOU064 and binder at a weight ratio of about 2:1.
E28. The method of E27 wherein the pharmaceutical composition comprises LOU064, binder and surfactant at a weight ratio of about 2:1:0.08.
E29. The method according to any one of embodiments E23 to E26, wherein the pharmaceutical composition comprises LOU064 and binder at a weight ratio of about 1:1.
E30. The method according to embodiment 29, wherein the pharmaceutical composition comprises LOU064, binder and surfactant at a weight ratio of about 1:1:0.05.
E31. The method according to any one of embodiments E23 to E30, wherein the pharmaceutical composition comprises LOU064, polyvinylpyrrolidone-vinyl acetate copolymer as a binder and sodium lauryl sulfate as a surfactant.
E32. The method according to any of the preceding embodiments wherein LOU064 is a crystalline form of the anhydrous free base characterized by an x-ray powder diffraction pattern comprising one or more representative peaks in terms of 2θ selected from the group consisting of 7.8±0.2 °2θ, 9.2±0.2 °2θ, 12.0±0.2 °2θ, 13.6±0.2 °2θ, 15.6±0.2 °2θ, 16.0±0.2 °2θ, 17.8±0.2 °2θ, 18.3±0.2 °2θ, 18.7±0.2 °2θ, 19.2±0.2 °2θ, 19.9±0.2 °2θ, 22.1±0.2 °2θ, 23.4±0.2 °2θ, 23.9±0.2 °2θ, 24.8±0.2 °2θ, 25.2±0.2 °2θ, 25.5±0.2 °2θ, 27.2±0.2 °2θ, and 29.6±0.2 °2θ, when measured at a temperature of about 25° C. and an x-ray wavelength, λ, of 1.5405 Å.

### DETAILED DESCRIPTION OF THE INVENTION

As used herein, Bruton's tyrosine kinase (BTK) is a cytoplasmic tyrosine kinase and member of the TEC kinase family. BTK is expressed in selected cells of the adaptive and innate immune system including B cells, macrophages, mast cells/basophils and thrombocytes. BTK is indispensable for signaling through the Fc epsilon receptor (FcεR1 for IgE) and the activating Fc gamma receptors (FcγR for IgG), as well as the B cell antigen receptor (BCR) and BTK inhibitors. BTK inhibitors like ibrutinib are approved for the treatment of B cell malignancies (Hendriks et al 2014). Recently, it has been demonstrated that inhibition of BTK leads to inhibition of mast cell and basophil activation/degranulation in vitro and to reduced wheal sizes in skin prick tests with patients suffering from IgE-mediated allergies (Smiljkovic et al 2017; Regan et al 2017; Dispenza et al 2018). Therefore, inhibition of BTK is an attractive therapeutic concept to treat various autoimmune and chronic inflammatory diseases, including rheumatoid arthritis, multiple sclerosis, systemic lupus erythematosus, chronic urticaria, atopic dermatitis, asthma, and primary Sjogren's Syndrome (Tan et al 2013; Whang and Chang 2014). Examples of BTK inhibitors include non-covalent, reversible BTK inhibitors such as fenebrutinib as well as covalent, irreversible inhibitors of BTK such as evobrutinib, tolebrutinib, rilzabrutinib, tirabrutinib, branebrutinib, orelabrutinib and remibrutinib (LOU064).

The term “comprising” encompasses “including” as well as “consisting,” e.g., a composition “comprising” X may consist exclusively of X or may include something additional, e.g., X+Y.

The phrase “pharmaceutically acceptable” as employed herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “about” in relation to a numerical value x means, for example, +/−10%. When used in front of a numerical range or list of numbers, the term “about” applies to each number in the series, e.g., the phrase “about 1-5” should be interpreted as “about 1-about 5”, or, e.g., the phrase “about 1, 2, 3, 4” should be interpreted as “about 1, about 2, about 3, about 4, etc.”

As used herein, the terms “salt” or “salts” refers to an acid addition or base addition salt of a compound for use in the method of the invention. “Salts” include in particular “pharmaceutically acceptable salts”. The term “pharmaceutically acceptable salts” refers to salts that retain the biological effectiveness and properties of the compounds of this invention and, which typically are not biologically or otherwise undesirable. In many cases, the compounds for use in the method of the inventions are capable of forming acid and/or base salts by virtue of the presence of amino group. Example of salts are those disclosed in WO 2015/079417 which is hereby incorporated by reference.

As used herein, the term “administering” in relation to a compound, e.g., LOU064, or another agent, is used to refer to delivery of that compound to a patient by any route, preferably by oral administration.

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical composition/formulation is contemplated.

The term “a therapeutically effective amount/dose” of a compound for use in the method of the invention refers to an amount of said compound that will elicit the biological or medical response of a subject, for example, reduction or inhibition of an enzyme or a protein activity, or ameliorate symptoms of HS, alleviate HS conditions, slow or delay disease progression of HS, or prevent HS.

The term “treatment” or “treat” is herein defined as the application or administration of LOU064 or a pharmaceutically acceptable salt thereof, or a pharmaceutical composition comprising LOU064 or a pharmaceutically acceptable salt thereof, to a subject or to an isolated tissue or cell line from a subject, where the subject has a particular disease (e.g., HS), a symptom associated with the disease (e.g., HS symptoms), or a predisposition towards development of the disease (if applicable), where the purpose is to cure (if applicable), reduce the severity of, alleviate, ameliorate one or more symptoms of the disease, improve the disease, reduce or improve any associated symptoms of the disease or the predisposition toward the development of the disease. The term “treatment” or “treat” includes treating a subject suspected to have the disease as well as subjects who are ill or who have been diagnosed as suffering from the disease or medical condition. In some aspect, the term “treating” refers to ameliorating the disease or disorder (i.e., slowing or arresting or reducing the development or progression of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. More specifically, the term “treating” the disease HS refers to treating the inflammatory lesions in HS patients (in numbers or quality or reducing their volume and size), and/or treating the abscesses and inflammatory nodules and/or draining fistulae in HS patients, and/or decreasing the amount of scarring and/or relieving the functional limitations associated with scarring. Treating the disease HS also refers to alleviating the pain, fatigue and/or itching associated with HS, reducing pus release and reducing the odor associated with pus release, and/or improving the quality of life and/or reducing the work impairment for HS patients.

As used herein, the term “prevention”, “prevent”, “preventing” of a disease or disorder (HS) refers to the prophylactic treatment of the disease or disorder; or delaying and or suppressing the onset or progression of the disease or disorder. More specifically, the term “preventing” the disease HS refers to preventing HS flares and or new lesions to appear; preventing scarring and preventing functional limitations associated with scarring and/or in particular preventing surgical interventions for HS.

As used herein, the phrase “population of subjects” is used to mean a group of subjects which would benefit biologically, medically or in quality of life from such treatment.

As used herein, the term “subject” refers to an animal. Typically, the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In a preferred embodiment, the subject is a human. The term “subject” is used interchangeably with “patient” when it refers to human.

As used herein, the phrases “has not been previously treated with a systemic treatment for HS” and “naïve” refer to an HS patient who has not been previously treated with a systemic agent, e.g., methotrexate, cyclosporine, or with a biological agent (such IL-12 and IL-23 blocking agents such as ustekinumab and guselkumab or with a TNF-alpha inhibitors such as adalimumab or infliximab, or with an IL-17 blocking agent such as secukinumab, ixekizumab and brodalumab) for HS. Systemic agents (i.e., agents given orally, by injection, etc.) differ from local agents (e.g., topicals and phototherapy) in that systemic agents have a systemic (whole body) effect when delivered to a patient. In some embodiments of the disclosed methods, regimens, uses, kits, and pharmaceutical compositions, the patient has not been previously administered a systemic treatment for HS.

As used herein, the phrase “has been previously treated with a systemic agent for HS” is used to mean a patient that has previously undergone HS treatment using a systemic agent. Such patients include those previously treated with biologics, such IL-12 and IL-23 blocking agents such as ustekinumab and guselkumab or with a TNF-alpha inhibitors such as Infliximab, or with an IL-17 blocking agent such as secukinumab, ixekizumab and brodalumab, and those previously treated with non-biologics, such as with a systemic immunosuppressant or immunomodulators (e.g. cyclosporine, methotrexate and cyclophosphamide), with systemic treatment including retinoids (such as isotretinoin), dapsone, metformin and oral zinc treatment. In some embodiments of the disclosure, the patient has been previously administered a systemic agent for HS. In some embodiments, the patient has been previously administered a systemic agent for HS (e.g., methotrexate, cyclosporine), but the patient has not been previously administered a systemic biological drug (i.e., a drug produced by a living organism, e.g., antibodies, receptor decoys, etc.) for HS (e.g., secukinumab, ustekinumab, ixekizumab, brodalumab, TNF alpha inhibitors (etanercept, adalimumab, infliximabremicade, etc.). In this case, the patient is referred to as “biological-naïve.” In some embodiments, the patient is biological-naïve.

As used herein, “selecting” and “selected” in reference to a patient is used to mean that a particular patient is specifically chosen from a larger group of patients on the basis of (due to) the particular patient having a predetermined criteria. Similarly, “selectively treating” refers to providing treatment to a patient having a particular disease, where that patient is specifically chosen from a larger group of patients on the basis of the particular patient having a predetermined criterion. Similarly, “selectively administering” refers to administering a drug to a patient that is specifically chosen from a larger group of patients on the basis of (due to) the particular patient having a predetermined criterion. By selecting, selectively treating and selectively administering, it is meant that a patient is delivered a personalized therapy based on the patient's personal history (e.g., prior therapeutic interventions, e.g., prior treatment with biologics), biology (e.g., particular genetic markers), and/or manifestation (e.g., not fulfilling particular diagnostic criteria), rather than being delivered a standard treatment regimen based solely on the patient's membership in a larger group. Selecting, in reference to a method of treatment as used herein, does not refer to fortuitous treatment of a patient having a particular criterion, but rather refers to the deliberate choice to administer treatment to a patient based on the patient having a particular criterion. Thus, selective treatment/administration differs from standard treatment/administration, which delivers a particular drug to all patients having a particular disease, regardless of their personal history, manifestations of disease, and/or biology. In some embodiments, the patient is selected for treatment based on having HS.

As used herein, the term “a,” “an,” “the” and similar terms used in the context of the present invention (especially in the context of the claims) are to be construed to cover both the singular and plural unless otherwise indicated herein or clearly contradicted by the context.

The term “pharmaceutical combination” as used herein means a product that results from the use or mixing or combining of more than one active ingredient. It should be understood that pharmaceutical combination as used herein includes both fixed and non-fixed combinations of the active ingredients. The term “fixed combination” means that the active ingredients, e.g., a compound of formula (I) or a pharmaceutically acceptable salt thereof, and one or more combination partners, are administered to a patient simultaneously as a single entity or dosage form. The term in such case refers to a fixed dose combination in one unit dosage form (e.g., capsule, tablet, or sachet). The terms “non-fixed combination” or a “kit of parts” both mean that the active ingredients, e.g., a compound of the present disclosure and one or more combination partners and/or one or more co-agents, are administered or co-administered to a patient independently as separate entities either simultaneously, concurrently or sequentially with no specific time limits wherein such administration provides therapeutically effective levels of the two compounds in the body of the patient, especially where these time intervals allow that the combination partners show a cooperative, e.g., an additive or synergistic effect. The term “non-fixed combination” also applies to cocktail therapy, e.g., the administration of three or more active ingredients. The term “non-fixed combination” thus defines especially administration, use, composition or formulation in the sense that the compounds described herein can be dosed independently of each other, i.e., simultaneously or at different time points. It should be understood that the term “non-fixed combination” also encompasses the use of a single agent together with one or more fixed combination products with each independent formulation having distinct amounts of the active ingredients contained therein. It should be further understood that the combination products described herein as well as the term “non-fixed combinations” encompasses active ingredients (including the compounds described herein) where the combination partners are administered as entirely separate pharmaceutical dosage forms or as pharmaceutical formulations that are also sold independently of each other. Instructions for the use of the non-fixed combination are or may be provided in the packaging, e.g., leaflet or the like, or in other information that is provided to physicians and/or medical staff. The independent formulations or the parts of the formulation, products, or compositions, can then be administered simultaneously or chronologically staggered, that is the individual parts of the kit of parts can each be administered at different time points and/or with equal or different time intervals for any part of the kit of parts. Particularly, the time intervals for the dosing are chosen such that the effect on the treated disease with the combined use of the parts is larger/greater than the effect obtained by use of only compound of Formula (I) or a pharmaceutically acceptable salt thereof; thus the compounds used in pharmaceutical combination described herein are jointly active. The ratio of the total amounts of a compound of formula (I) or a pharmaceutically acceptable salt thereof, to a second agent to be administered as a pharmaceutical combination can be varied or adjusted in order to better accommodate the needs of a particular patient sub-population to be treated or the needs of the single patient, which can be due, for example, to age, sex, body weight, etc. of the patients.

The terms “co-administration” or “combined administration” or the like as utilized herein are meant to encompass the administration of one or more compounds described herein together with a selected combination partner to a single subject in need thereof (e.g., a patient or subject), and are intended to include treatment regimens in which the compounds are not necessarily administered by the same route of administration and/or at the same time.

### LOU064

LOU064 (═N-(3-(6-amino-5-(2-(N-methylacrylamido)ethoxy) pyrimidin-4-yl)-5-fluoro-2-methylphenyl)-4-cyclopropyl-2-fluorobenzamide, INN: remibrutinib) is disclosed in WO 2015/079417 Å1 as a drug candidate for the selective inhibition of Bruton's tyrosine kinase. This compound is a potent, highly selective, irreversible covalent BTK inhibitor. Due to binding to an inactive conformation of BTK, LOU064 exhibits an exquisite kinase selectivity and, thus, reduces kinase off-target binding and due to covalent inhibition, the compound exhibits a potent and sustained pharmacodynamic effect without the need for extended and high systemic compound exposure (Angst, D. et al., Discovery of LOU064 (Remibrutinib), a Potent and Highly Selective Covalent Inhibitor of Bruton's Tyrosine Kinase, J Med Chem. 2020 May 28; 63(10):5102-5118).

LOU064 for use in the methods of the invention, is the free base as represented by Formula (I):

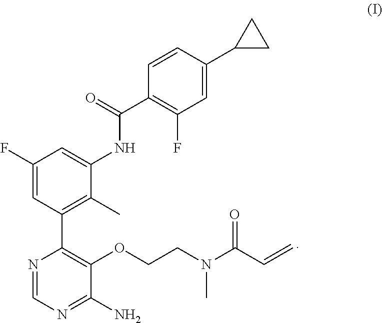 [SMILES: C=CC(=O)N(C)CCOc1c(N)ncnc1-c1cc(F)cc(NC(=O)c2ccc(C3CC3)cc2F)c1C]

In other embodiment, N-(3-(6-amino-5-(2-(N-methylacrylamido)ethoxy) pyrimidin-4-yl)-5-fluoro-2-methylphenyl)-4-cyclopropyl-2-fluorobenzamide is the anhydrous crystalline form A of the free base as disclosed in WO2020/234779 (Example 1), which is thereby incorporated by reference.

LOU064, which has previously been suggested for use in the treatment of chronic spontaneous urticaria (CSU) (WO2020/234782 Å1) and Sjögren's Syndrome (SjS) (WO2020/234781 Å1), is currently being tested in phase II clinical studies for CSU and SjS.

In WO2020/234782 Å1 b.i.d. administration of doses of 10 mg, 25 mg and 100 mg were generally suggested to reach maximal efficacy in CSU.

In a Phase 2b, randomized, double-blind, placebo-controlled trial evaluating the efficacy and safety of LOU064 over 12 weeks in patients inadequately controlled by Hr-antihistamines, with at least moderately active chronic spontaneous urticaria (CSU), patients received LOU064 10 mg q.d. (once daily), 35 mg q.d., 100 mg q.d., 10 mg b.i.d. (twice daily), 25 mg b.i.d., 100 mg b.i.d., or placebo (1:1:1:1:1:1:1 ratio). The 25 mg b.i.d. regimen was found to be particularly effective compared to the other doses.

Hence, a dose of 25 mg b.i.d. was selected for a subsequent phase Ill clinical study for CSU.

BTK occupancy in blood and/or tissues has been reported to be a suitable biomarker for selecting doses for clinical studies such as CSU and SjS studies (WO2020/234782 and WO2020/234781).

Furthermore, it has been reported that BTK occupancy and duration of BTK occupancy is different in blood and in various tissues in female rat (WO2020/234781).

BTK occupancy in different tissues is relevant to efficacy and optimum dosage selection in different indications. However, there is no currently agreed complete picture of all tissues that are relevant to the HS indication and therefore which tissue(s) need to be penetrated for treating or preventing HS. As reported in rat, the BTK occupancy and BTK occupancy half-life is different in blood and in various tissues.

BTK occupancy half-life is dependent on the turnover rates (ability of the BTK cells to regenerate). Such turnover rates differ in each tissue and are species specific. The BTK occupancy is further dependent on the PK/PD properties of a compound which is also species dependent.

BTK inhibitors may act or intervene through three basic mechanisms of action or pathways:
1) reduction of allergic/type I hypersensitivities through FcεR1,
2) inhibition of B cell autoreactivity and immune complexes in autoreactivity/inflammation or type 3 hypersensitivity through FcγR an/or the BCR,
3) inhibition of auto-antibody secretion in Auto-antibody pathologies or type 2 hypersensitivity through the BCR
   Without being linked to a theory, BTK inhibition is linked to the latter 2 pathways, as HS has been suggested to be linked to B-cell activity (Rumberger et al. 2020, Gudjonsson et al.) as well as through the presence of auto-antibodies (Byrd et al (2018), J. Dermatol.; 179(3):792-794, (2019), Camona-Rivera et al. (2021) J Invest Dermatol. October 1:S0022-202X(21)02286-7. doi: 10.1016/j.jid.2021.07.187). B-cell and plasma cells can be found in tertiary lymphoid organs in lesional tissue. BTK turn-over is potentially higher in B cells located in tertiary lymphoid organs of HS lesions compared to skin mast cells and sufficient target tissue penetration might be more difficult to achieve in HS compared to CSU, due to the fibrotic tissue remodeling as well as abscess and fistula tract.
   CSU, on the other hand, is likely to depend in particular to the first above mentioned pathway, as demonstrated by the activity of anti-IgE antibodies (via FcεR1), Therefore, clinical response in HS may not be expected using the CSU dose or any doses disclosed for other indications.

Therefore, without being bound to any theory, an efficacious dose of LOU064 for the treatment or prevention of HS is a dose which allows higher tissue concentration into inflamed, partially fibrotic HS lesions with potential higher BTK turnover.

### Pharmaceutical Composition for Use in the Methods of the Invention

The BTK inhibitor, i.e., compound of Formula (I), or a pharmaceutically acceptable salt thereof, may be used as a pharmaceutical composition when combined with a pharmaceutically acceptable carrier. Such a composition may contain, in addition to the compound of Formula (I) or a pharmaceutically acceptable salt thereof, carriers, various diluents, fillers, salts, buffers, stabilizers, solubilizers, and other materials known in the art. The characteristics of the carrier depends on the route of administration. The pharmaceutical compositions for use in the disclosed methods may also contain additional therapeutic agents for treatment of the particular targeted disorder. For example, a pharmaceutical composition may also include anti-inflammatory or anti-itch agents. Such additional factors and/or agents may be included in the pharmaceutical composition to produce a synergistic effect with the compound of Formula (I) or a pharmaceutically acceptable salt thereof, or to minimize side effects caused by the compound of Formula (I) or a pharmaceutically acceptable salt thereof. In preferred embodiments, the pharmaceutical composition for use in the disclosed methods comprise compound of Formula (I) or a pharmaceutically acceptable salt thereof, in a daily dose of about 50 mg to 200 mg, e.g. 25 mg bid or 100 mg bid.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients, which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Pharmaceutical compositions for use in the disclosed methods may be manufactured in conventional manner. In one embodiment, the pharmaceutical composition is provided for oral administration. For example the pharmaceutical compositions are tablets or gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Therefore, pharmaceutical composition for use in the methods of the invention comprises LOU064 and one or more pharmaceutically acceptable carriers, each of which is independently selected from a filler, a lubricant, a binder, a desintegrant and a glidant.

In another embodiment, a suitable pharmaceutical composition, LOU064 may be present in any pharmaceutically acceptable form. It may be preferable that the pharmaceutical composition is in tablet or capsule form. Tablets may be either film coated or enteric coated according to methods known in the art. It may also be preferable to include LOU064 in the pharmaceutical composition/formulation as nanosized or as microsized particles.

If LOU064 is present in the pharmaceutical formulation in the form of nanosized particles, the mean particle size can be less than 1000 nm. Preferably, the mean particle size of LOU064 can be less than 500 nm, more preferably less than 250 nm.

In a preferred embodiment, the mean particle size of LOU064 can be between about 50 nm and about 1000 nm, or between about 50 nm and about 750 nm, or between about 60 nm and about 500 nm, or between about 70 nm and about 350 nm, or between about 100 nm and about 170 nm, More preferably, the mean particle size of LOU064 may be between about 100 nm and about 350 nm, or between about 110 nm and about 200 nm, or between about 120 nm and about 180 nm or between about 120 nm and about 160 nm, preferably the mean particle size of LOU064 can be about 150 nm to about 200 nm.

If LOU064 is present in the pharmaceutical formulation in the form of nanosized particles, oral administration is preferably at a dose of about 25 mg to about 100 mg twice daily, more preferably at a dose of about 100 mg twice daily.

If LOU064 is present in the pharmaceutical formulation in the form of microsized particles, the mean particle size can be 1-5 μm or preferably 1.0-1.5 μm. Preferably, the mean particle size of LOU064 can be 1.1 to 1.3 μm.

If LOU064 is present in the pharmaceutical formulation in the form of microsized particles, oral administration is preferably at a dose of about 25 mg to about 100 mg twice daily, for example at a dose of about 25 mg twice daily.

In a preferred embodiment the polydispersity index (PI) is between 0.01 and 0.5, more preferably between 0.1 and 0.2, in particular 0.12-0.14. A preferred particle size distribution is shown in FIG. 5.

The above-mentioned mean particle sizes are intensity weighted. The mean particle size can be determined by means of dynamic light scattering. Preferably, the mean particle size is determined by Photon Correlation Spectroscopy (PCS). In particular, for determining the mean particle size the device “Zetasizer Nano ZS”, Version 7.13 from Malvern Panalytical Ltd., UK can be used.

Preferably, the measurement is carried out as wet dispersion method using 0.1 mM NaCl solution in purified water (1:10), wherein the attenuator index is 2-9, in particular 5. The measurement is preferably carried out at 25° C. Further preferred settings of the measurement systems are as follows:
Cell: Disposable sizing cuvette
Count Rate (kcPs): 315
Duration: 60 sec
Measurement Position (mm): 4.65.

In one embodiment of the invention, a LOU064 composition is formulated in accordance with routine procedures as a pharmaceutical composition adapted for oral administration to human beings. Typically, compositions for oral administration are capsules or tablets.

In one embodiment, a pharmaceutical composition/formulation for LOU064 can be formulated according to a formulation disclosed in U.S. application No. 63/141,558 or its family members (published PCT WO2022/162513), herein incorporated by reference.

According to the invention, a suitable pharmaceutical composition for oral administration comprises LOU064 and binder.

Suitable binders include polyvinylpyrrolidone-vinyl acetate copolymer, polyvinyl pyrrolidone, hydroxypropyl cellulose, hydroxypropylmethyl cellulose, hypromellose, carboxymethyl cellulose, methyl cellulose, hydroxyethyl cellulose, carboxyethyl cellulose, carboxymethylhydroxyethyl cellulose, polyethylene glycol, polyvinylalcohol, shellac, polyvinyl alcohol-polyethylene glycol co-polymer, polyethylene-propylene glycol copolymer, or a mixture thereof. Preferably the binder is polyvinylpyrrolidone-vinyl acetate copolymer.

The weight ratio of LOU064 and binder can be from about 3:1 to about 1:3; e.g. about 3:1, about 2:1, about 1:1, preferably the weight ratio of LOU064 and binder is about 2:1 or about 1:1.

Preferably, a suitable pharmaceutical composition for oral administration comprises LOU064, binder and surfactant.

Suitable surfactants include sodium lauryl sulfate, potassium lauryl sulfate, ammonium lauryl sulfate, sodium lauryl ether sulfate, polysorbates, perfluorobutanesulfonate, dioctyl sulfosuccinate, or a mixture thereof. Preferably the surfactant is sodium lauryl sulfate.

The weight ratio of LOU064, binder and surfactant is about 2:1:0.5, or about 2:1:0.1, or about 2:1:0.08, or about 2:1:0.05, or about 2:1:0.04, or about 2:1:0.03, or about 2:1:0.02. Preferably, the weight ratio of LOU064, binder and surfactant is about 2:1:0.08 or about 1:1:0.05.

In a particularly preferred embodiment, a suitable pharmaceutical composition for oral administration comprises LOU064, binder and surfactant, wherein the binder is polyvinylpyrrolidone-vinyl acetate copolymer (copovidone) and the surfactant is sodium lauryl sulfate (SLS), and wherein the weight ratio of LOU064, copovidone and SLS is about 2:1:0.08. It is further particularly preferred that LOU064 is present in this pharmaceutical composition in the form of nanosized particles, preferably having a mean particle size as measured by PCS of between about 100 nm and about 200 nm.

### Methods of Treatment or Prevention of HS and Using LOU064 or a Pharmaceutically Acceptable Salt Thereof, or a Crystalline Form Thereof.

Disclosed methods of, and LOU064 or a pharmaceutically acceptable salt thereof for use in treating or preventing HS, in a subject in need thereof, comprising herein are administering the subject a therapeutically effective dose of LOU064 or a pharmaceutically acceptable salt thereof. LOU064 or a pharmaceutically acceptable salt thereof for use in the disclosed methods, may be used in vitro, ex vivo, or incorporated into pharmaceutical compositions and administered in vivo to treat HS patients (e.g., human patients).

The appropriate dosage will vary depending upon, for example, the particular pharmaceutically acceptable salt of LOU064, the particular polymorphic form of LOU064, patient's weight, the mode of administration, the pharmaceutical composition, and the nature and severity of the condition being treated, and on the nature of prior treatments that the subject has undergone. Ultimately, the attending health care provider will decide the amount of LOU064 with which to treat each individual subject. In some embodiments, the attending health care provider may administer low doses of LOU064 and observe the subject's response.

In one embodiment of the present disclosure, LOU064 or a pharmaceutically acceptable salt thereof, is administered orally at a dose of about 50 mg to about 200 mg daily.

In one embodiment of the present disclosure, LOU064 or a pharmaceutically acceptable salt thereof is administered orally at a dose of about 25 mg twice daily to about 100 mg twice daily, e.g about 25 mg twice daily (BID), about 50 mg BID or about 100 mg BID.

In another embodiment, LOU064 is administered orally at a dose of about 25 mg twice daily.

In another embodiment, LOU064 is administered orally at a dose of about 100 mg twice daily.

The duration of therapy using a pharmaceutical composition of the present disclosure will vary, depending on the severity of the disease or disorder to be treated and the condition and personal response of each individual subject. In some embodiments, the subject is administered LOU064 for a short-term e.g. up to 1 week, e.g. up to 2 weeks, e.g. up to 4 weeks, e.g. up to 12 weeks, e.g. up to 16 weeks, e.g. up to 24 weeks.

In some other embodiments, the subject is preferably administered LOU064 for a long-term (e.g. LOU064 is used without restriction in a total duration for as long as the disease is present justifying its use, e.g. at least 6 months, e.g. for more than 1 year, 2 years, 3 years, 4, years, 10 years. LOU064 or a pharmaceutically acceptable salt thereof might be used up to 5 years, 10 years, 15 years, 20 years or for life. In a preferred embodiment, the treatment with LOU064 according to the invention, is a chronic treatment.

HS is a chronic, inflammatory, scarring condition involving primarily the intertriginous skin of the axillary, inguinal, inframammary, genito-anal, and perineal areas of the body. It is also referred to as acne inversa. Three diagnostic criteria establish a diagnosis of HS: typical lesions (deep-seated painful nodules [blind] boils in early primary lesions, or abscesses, draining sinuses, bridged scars, and “tombstone” open comedones in secondary lesions); typical topography (axillae, groin, genitals, perineal and perianal regions, buttocks, and infra- and intermammary areas; and chronicity and recurrence (Margesson and Danby (2014) Best Practices and Res. Clin. Ob. And Gyn 28:1013-1027). The physical extent of HS can be classified using Hurley's clinical staging, shown below in Table 1:

TABLE 1
Hurley's Stages of HS. Practically speaking, a
patient having Hurley's stage III may have burned-
out Stage III, but active Stage I or II lesions.

| | |
|---|---|
| Stage I | Abscesses only (single or multiple) without |
| | sinus tracts and cicatrization (scarring) |
| Stage II | Abscesses (single or multiple) with tract |
| | formation or cicatrization, single or |
| | multiple widely separated lesions |
| | (e.g., >10 cm apart) |
| Stage III | Diffuse or near diffuse involvement, |
| | or multiple interconnecting tracts or |
| | abscesses across entire area |

HS consists of follicular plugging, ductal rupture, and secondary inflammation. Patients first experience a plug in the follicular duct, which, over time leads to duct leak and horizontal rupture into the dermis. When repair of the folliculo-pilosebaceous (FPSB) fails, the follicular fragments stimulate three reactions that begin the HS disease course. The first is an inflammatory response, triggered by the innate immune system, causing purulence and tissue destruction, and leading to foreign body reactions and extensive scarring. The second reaction leads to epithelialized sinuses, which may evolve from stem cells derived from the FPSB unit that survive the destruction caused by the inflammatory response. Third, an invasive proliferative gelatinous mass is produced in most cases, consisting of a gel containing inflammatory cells, and, it is postulated, the precursors of the epithelialized elements described above. (See Margesson and Danby (2014). As used herein, the phrase “slowing HS disease progression” means decelerating the advancement rate of any of the aspects of the HS disease course described above, particularly the inflammatory response. In some embodiments of the disclosure, treatment with LOU064 slows HS disease progression.

Recurrence of HS in a patient includes the development of papules, pustules or inflammatory nodules, pain and itching, abscesses, draining, and any combination thereof. As used herein, “HS flare” (and the like) is defined as at least a 25% increase in abscesses and inflammatory nodule counts (AN), with a minimum increase of two ANs relative to a baseline.

In some embodiments of the disclosure, treatment according to the disclosed methods with LOU064 prevents HS flares, decreases the severity of HS flares, and/or decreases the frequency of HS flares. In some embodiments, when a population of HS patients is treated according to the disclosed methods, less than 5%, less than 10%, less than 15% or less than 20% experiences a flare during the first 16 weeks of treatment.

As used herein, the phrase “decreasing the severity of HS flares” and the like means reducing the intensity of an HS flare, e.g., reducing the number and/or size of abscesses and/or inflammatory nodules, reducing the strength of a particular flare component (e.g., reducing the number, size, thickness, etc. of abscesses and/or inflammatory nodules, reducing the extent of skin irritation (itching, pain) etc.), and/or reducing the amount of time a flare (or component thereof) persists.

As used herein, the phrase “decreasing the frequency of HS flares” and the like means reducing the incidence of HS flares, e.g., reducing the incidence of abscesses and/or inflammatory nodules. By decreasing the frequency of HS flares, a patient will experience fewer HS relapses. The incidence of flares may be assessed by monitoring a patient over time to determine if the prevalence of flares decreases.

As used herein, the phrase “preventing HS flares” means eliminating future HS flares and/or flare components.

The effectiveness of an HS treatment may be assessed using various known methods and tools that measure HS disease state and/or HS clinical response. Some examples include, e.g., Hurley's staging, severity assessment scoring system (SAHS), a Sartorius score, a modified Sartorius score, the HS physicians' global assessment (HS-PGA) score, a visual analog scale (VAS) or numeric rating scale (NRS) to rate skin related pain or skin itch, the dermatology life quality index (DLQI), HS clinical response based on sum of abscesses and inflammatory nodules (HiSCR: HiSCR50, HiSCR75 or HiSCR90), simplified HiSCR, EuroQuol-5D (EQ5D), hospital anxiety and depression scale, healthcare resources utilization, Hidradenitis Suppurativa Severity Index (HSSI), Work productivity index (WPI), inflamed body surface area (BSA), Acne Inversa Severity Index (AISI) etc. (see, e.g., Deckers and Prens (2016) Drugs 76:215-229; Sartorius et al. (2009) Br. J. Dermatol 161:831-39; Chiricozzi et al. (2015) Wounds 27(10):258-264). In some embodiments, the effectiveness of the method of the invention disclosed herein may be assessed by the HS physicians' global assessment (HS-PGA), severity assessment scoring score (SAHS), score numeric rating scale (NRS) (itch or pain), the dermatology life quality index (DLQI), HS clinical response based on sum of abscesses and inflammatory nodules (HiSCR i.e HiSCR50, HiSCR75 or HiSCR90), and/or simplified HiSCR. Preferably, the effectiveness of the HS treatment as disclosed herein may be assessed by HS clinical response based on sum of abscesses and inflammatory nodules (HiSCR i.e HiSCR50, HiSCR75 or HiSCR90), and/or simplified HiSCR.

In some embodiments, an HS patient achieves a HiSCR in response to HS treatment. In some embodiments, when a population of HS patients is treated according to the disclosed methods, at least 30%, at least 40%, at least 50%, at least 60% or at least 70% achieve by week 16 at least one of the following:
HiSCR50;
HiSCR75;
HiSCR90; or
Simplified HiSCR (sHiSCR); preferably 2HiSCR.

In other embodiments, the effectiveness of the HS treatment as disclosed herein can be measured by the difference between the responder rate in the treated patients (i.e. patient achieving a HiSCR response (HiSCR i.e HiSCR50, HiSCR75, HiSCR90 or sHiSCR response) to the HS treatment with compound of the invention) and the responder rate in the placebo treated patients, by week 16 of treatment. In some embodiment, this difference in the responder rate as measured by HiSCR (HiSCR50, HiSCR75, HiSCR90 or sHiSCR) is at least 15%, at least 25%, at least 30% or at least 35%.

Preferred scoring systems for treatment response are the HiSCR (HiSCR50, HiSCR75 or HiSCR90), simplified HiSCR, pain or skin itch NRS (e.g. pain NRS30 or skin itch NRS30), severity assessment scoring system (SAHS), HS-PGA, inflammatory lesion count (count of abscesses, inflammatory nodules, and/or draining fistulae), ISH4 and DLQI.

The Hidradenitis Suppurativa Clinical Response (HiSCR) is a measure of clinical response to HS treatment. A HiSCR50 response to treatment (compared to baseline) is as follows: 1) at least 50% reduction in abscesses and inflammatory nodules (AN50), and 2) no increase in the number of abscesses, and 3) no increase in the number of draining fistulae.

In some embodiments, an HS patient achieves a HiSCR50 in response to HS treatment.

In some embodiments, when a population of HS patients is treated according to the disclosed methods, at least 40%, at least 50%, at least 60%, or at least 70% achieve a HiSCR50 by week 16 of treatment.

As used herein the “simplified HiSCR” or “sHiSCR” refers to a modified HiSCR that does not include the abscess count versus baseline when assessing progression of lesions. In preferred embodiments, an HS patient achieves a simplified HiSCR in response to HS treatment. In some embodiments, when a population of HS patients is treated according to the disclosed methods, at least 40%, at least 50%, at least 60%, or at least 70% achieve a simplified HiSCR by week 16 of treatment.

A HiSCR75 response to treatment (compared to baseline) is as follows: 1) at least 75% reduction in abscesses and inflammatory nodules, and 2) no increase in the number of abscesses, and 3) no increase in the number of draining fistulae.

A HiSCR90 response to treatment (compared to baseline) is as follows: 1) at least 90% reduction in abscesses and inflammatory nodules, and 2) no increase in the number of abscesses, and 3) no increase in the number of draining fistulae.

In preferred embodiments, an HS patient achieves a HiSCR75 or a HiSCR90 in response to HS treatment.

In some embodiments, when a population of HS patients is treated according to the disclosed methods, at least 40%, at least 50%, at least 60%, or at least 70% achieve a HiSCR75 by week 16 of treatment.

In some embodiments, when a population of HS patients is treated according to the disclosed methods, at least 40%, at least 50%, at least 60%, or at least 70% achieve a HiSCR90 by week 16 of treatment.

Pain can be assessed using a numeric rating scale (NRS). In some embodiments, an HS patient achieves an improved pain NRS in response to HS treatment. Pain NRS30 is defined as at least 30% reduction in pain and at least 1 unit reduction from baseline in Patient's Global Assessment (PGA) of Skin Pain from baseline in patients with a baselines score of 3 or higher. In some embodiments, an HS patient achieves an NRS30 in response to HS treatment.

In some embodiments, when a population of HS patients is treated according to the disclosed methods, at least 25%, at least 30%, at least 40%, at least 50%, or at least 60% achieve an NRS30 by week 16 of treatment. In a preferred aspect of this embodiment, when a population of HS patients is treated according to the disclosed methods, at least 30% achieve an NRS30 by week 16 of treatment. In some embodiments, in response to treatment according to the claimed methods, the patient experiences reduction in pain, as measured by VAS or NRS, preferably NRS, as early as 1 or 2 weeks after initial dosing.

The severity assessment scoring system (SAHS) is described in Hassam et al. JAMA Dermatol (2018), 154(3): 330-335. The severity of HS can be assessed by the SAHS score, for which the following items are surveyed: number of involved regions (axilla left, axilla right, submammary left, submammary right, intermammary or chest, abdominal, mons pubis, groin left, groin right, genital, perianal or perineal, gluteal left, gluteal right, and others [eg, neck, retroauricular]), number of inflammatory and/or painful lesions other than fistula (ILOF), and number of fistula. These physician-rated items were completed by 2 patient-reported items: patients were asked for number of new boils or number of existing boils, which flared up during the past 4 weeks and to rate the current severity of pain (NRS) of the most symptomatic lesion in the course of their daily activities (e.g., sitting, moving, or working) on a numerical rating scale. The SAHS score is a composite score of all the collected information above. A mild case of HS is defined by a SAHS score of 4 or less. A moderate HS is defined by a SASH score of 5 to 8, and a severe case of HS is defined by SASH of 9 or higher.

In some embodiments, an HS patient achieves an improved SAHS score in response to HS treatment. In some embodiments, an HS patient achieves at least one point reduction from baseline in the SAHS score in response to HS treatment. In other embodiments, an HS patient achieves at least two points reduction or at least 3 points reduction from baseline in the SAHS score in response to HS treatment. Preferably, the SAHS score was at least 4 at baseline before treatment with LOU064.

The International Hidradenitis Suppurativa Severity Score System (IHS4) is a clinically validated tool to dynamically assess HS severity, that can be used in clinical trials or in real-life conditions (Zouboulis et al. (2017); Br. J. Dermatol.; 177(5): 1401-9.). The severity of HS according to this tool is assessed by adding up the individual HS lesions following this scheme:

$$number of nodules \times 1 + number of abscesses \times 2 + number of draining tunnels ( fistulae / sinuses ) \times 4$$

The severity of HS is then defined as:
Mild HS: ≤3 points
Moderate HS: 4-10 points
Severe HS: ≥11 point
   The definition of the HS lesions are as follows: A nodule (inflammatory nodule) is a raised, three-dimensional, round, infiltrated lesion with a diameter of >10 mm. An abscess is a tender but fluctuating mass with a diameter of >10 mm, surrounded by an erythematous area; the middle of an abscess contains pus. A draining tunnel is a raised, tender but fluctuating longitudinal mass of variable length and depth, ending at the skin surface, and sometimes oozing a fluid. Fistulae and sinuses are examples of tunnels.

In some embodiment, an HS patient achieves an improved IHS4 score (i.e. decrease) In some embodiments, an HS patient achieves at least two point reduction from baseline in the IHS4 score in response to HS treatment. In other embodiments, an HS patient achieves at least 3 points reduction or at least 4 points reduction from baseline in the IHS4 score in response to HS treatment.

The DLQI is the most established dermatological life quality instrument. It consists of questions regarding the impact of the skin disease on feelings and different aspects of daily life activities during the last week. Each question is scored from 0 (not at all) to 3 (very much). A total of 30 points is the maximum score, where 0-1 is regarded as no effect, 2-5 small, 6-10 moderate, 11-20 very large and 21-30 as extremely large effect on the patient's life. (See Finlay and Khan (1994) Clin Exp Dermatol 19:210-16). In some embodiments, an HS patient achieves an improved DLQI in response to HS treatment.

In some embodiments, in response to treatment according to the claimed methods, the patient experiences reduction in CRP, as measured by standard CRP assay or a high sensitivity CRP (hsCRP) assay, as early as 1 or 2 weeks after initial dosing. As used herein, “C-reactive protein” and “CRP” refer to serum C-reactive protein, a plasma protein commonly used as an indicator of the acute phase response to inflammation. The level of CRP in plasma may be given in any concentration, e.g., mg/dl, nmol/L. Levels of CRP may be measured by a variety of standard assays, e.g., radial immunodiffusion, electroimmunoassay, immunoturbidimetry, ELISA, turbidimetric methods, fluorescence polarization immunoassay, and laser nephelometry. Testing for CRP may employ a standard CRP test or a high sensitivity CRP (hs-CRP) test (i.e., a high sensitivity test that is capable of measuring low levels of CRP in a sample using laser nephelometry). Kits for detecting levels of CRP may be purchased from various companies, e.g., Calbiotech, Inc, Cayman Chemical, Roche Diagnostics Corporation, Abazyme, DADE Behring, Abnova Corporation, Aniara Corporation, Bio-Quant Inc., Siemens Healthcare Diagnostics, etc.

The Sartorius HS score (also called the HS score, or HSS) is made by counting involved regions, nodules, and sinus tracts in an HS patient. (Sartorius et al. (2003) Br J Dermatol 149:211-13). The modified Sartorius HS score is a revision of the original version of the HSS by making minor simplifications which made it more practical to use, e.g., fewer specific lesions to include in the score, changes to the number of points given for each parameter, etc. (Sartorius et al. (2009) Br. J Dermatol. 161:831-839). In some embodiments, an HS patient achieves an improved modified Sartorius HS in response to HS treatment.

The HS physicians' global assessment (HS-PGA) is a 6-scale evaluating scale (scores range from 0-5) based on the number of HS lesions (i.e., abscesses, draining fistulas, inflammatory nodules, and noninflammatory nodules). (Kimball A B, Kerdel F, Adams D et al Adalimumab for the treatment of moderate to severe hidradenitis suppurativa: a parallel randomized trial. Ann Intern Med 2012; 157: 846-855). In some embodiments, an HS patient achieves an improved HS-PGA in response to HS treatment. In some embodiments, an HS patient achieves at least a 2 points reduction from baseline in the HS-PGA score in response to HS treatment. Preferably, the HS-PGA score was at least 3 at baseline before treatment with a LOU064.

In some embodiments, when a population of HS patients are treated according to the disclosed methods, at least 40%, at least 50%, at least 60%, at least 70%, at least 80% or at least 90% of patients who have responded to treatment by week 16 (e.g., patients achieving a HiSCR (e.g. HiSCR50; HiSCR75 or HiSCR90) or simplified HiSCR by week 16) have sustained response 3 months after the end of treatment or 6 months after the end of treatment, or 12 months after the end of treatment. In another aspect of this embodiment, at least 40% of patients or at least 50% of patients who have responded to treatment by week 16 (e.g., patients achieving a HiSCR (e.g. HiSCR50; HiSCR75 or HiSCR90) or simplified HiSCR by week 16) have sustained response 3 months after the end of treatment. Preferably at least 70% of patients who have responded to treatment by week 16 (e.g., patients achieving a HiSCR (e.g. HiSCR50; HiSCR75 or HiSCR90) or simplified HiSCR by week 16) have sustained response 3 months after the end of treatment. As used herein the term “sustained” means that an outcome or goal (e.g., pain reduction, inflammation reduction) is substantially maintained for a given time.

The lesion-related itching can be assessed by a patient survey. The patient is asked to rate the lesion-related itching from 0 (no itching) to 10 (worse possible itching). In some embodiments, when a population of HS patients are treated according to the disclosed methods, the itching score improves by at least 2 points, preferably a least 3 points. Furthermore, when compared to the placebo group, a difference between the treated group and the placebo group is at least 1 point.

The lesion-related itching can also be assessed by the itch numerical scale (NRS). Itch is reported by the vast majority of HS patients (Fernandez et al (2021) Itch and pain by lesion morphology in hidradenitis suppurativa patients. Int J Dermatol.; 60(2):e56-e59). The NRS is a segmented numeric version of the visual analog scale (VAS) in which a respondent selects a whole number (0-10 integer on an 11-point scale) that best reflects the intensity of their skin disease related itch (Nguyen et al. (2021), J Eur Acad Dermatol Venereol. 2021 January; 35(1):50-61). In some embodiments, an HS patient achieves an improved skin NRS in response to HS treatment. Itch NRS30 is defined as at least 30% reduction in skin itch from baseline. In some embodiments, an HS patient achieves a skin NRS30 in response to HS treatment. In some embodiments, when a population of HS patients is treated according to the disclosed methods, at least 25%, at least 30%, at least 40%, at least 50%, or at least 60% achieve a skin NRS30 by week 16 of treatment. In a preferred aspect of this embodiment, when a population of HS patients is treated according to the disclosed methods, at least 30% achieve a skin NRS30 by week 16 of treatment. In some embodiments, in response to treatment according to the claimed methods, the patient experiences reduction in skin itch, as measured by skin NRS as early as 1 or 2 weeks after initial dosing.

The odor caused by the draining of the lesion can be assessed by a patient survey. The patient is asked to rate the odor caused by the draining of the lesion from 1 (no odor), 2 (a little odor), 3 (moderate odor) to 4 (a lot of odor). In some embodiments, when a population of HS patients are treated according to the disclosed methods, the itching score improves by at least 1 point, preferably a least 2 points. Furthermore, when compared to the placebo group, a difference between the treated group and the placebo group is at least 1 point.

The impact on HS on the ability to complete work can be assessed by a patient survey. The patient is asked to rate how much HS impacts the ability to complete work from 1 (no at all), 2 (a little), 3 (moderately), 4 (a great deal) to 5 (unable to do any work). In some embodiments, when a population of HS patients are treated according to the disclosed methods, the itching score improves by at least 1 point, preferably a least 2 points. Furthermore, when compared to the placebo group, a difference between the treated group and the placebo group is at least 1 point.

### Safety:

Short-term safety of LOU064 at single doses up to 600 mg and further at 100 mg b.i.d. for up to 18 days has been shown in Phase I clinical studies. However, no data concerning long-term safety (i.e. beyond 52 weeks) are available at this time.

Considering dose-limiting side effects observed with the covalent irreversible BTK inhibitors evobrutinib and tolebrutinib, with evobrutinib showing dose-limiting liver enzyme elevations already at a dose of 75 mg b.i.d. in Phase II clinical studies and tolebrutinib showing dose-limiting diarrhea (Becker A. et al., 2019, Clin Transl Sci; 13, 325-336; Montalban X. et al., 2019, N Engl J Med; 380(25): 2406-17, Smith P. F. et al., 2019, ACTRIMS Forum, Feb. 28, 2019, P072), it is encouraging that no meaningful increase of the incidence of these adverse events was seen with LOU064 even at the higher dose of 100 mg b.i.d. over an extended period of time (up to 52 weeks). In particular, LOU064 does not induce any dose-limiting liver enzyme elevations and other off-target-effects at a dose of 100 mg b.i.d. over an extended period of time (up to 52 weeks). LOU064 is therefore suited for long-term treatment.

Therefore, one object of the invention is LOU064 for use in the described methods, wherein by week 12, by week 24 or by week 52 of treatment the levels of alanine aminotransferase (ALT), aspartate aminotransferase (AST), and lipase do not change by more than 10% as compared to the baseline level at the start of therapy.

It was therefore particularly surprising that LOU064 not only efficiently treat HS but also possesses a better safety profile as compared to other BTK inhibitors, particularly as compared to acalabrutinib, especially when treatment is maintained over an extended period of time. For example, BTK inhibitors (ibrutinib, acalabrutinib, and zanubrutinib) which are currently approved primarily for the treatment of hematologic malignancies have known safety liabilities. Main safety liabilities include infections, effect on platelet function (risk for bleeding), and cytopenias. The other safety concerns for one or more approved BTKis include cardiac arrhythmia (atrial fibrillation and flutter) and, for ibrutinib only, cardiac failure and hypertension.

Hence, in a preferred embodiment of the present invention, LOU064 or a pharmaceutically acceptable salt thereof for use in treating HS is used in a long-term treatment. The term long-term treatment indicates that LOU064 or a pharmaceutically acceptable salt thereof is used over an extended period of time. For example, LOU064 or a pharmaceutically acceptable salt thereof can be used safely for more than 6 months, 10 months, 1 years, 2 years, 3 years, 4, years, 10 years. LOU064 or a pharmaceutically acceptable salt thereof might be used up to 2 years, 5 years, 10 years, 15 years, 20 years or for life.

In one embodiment, LOU064 not only efficiently treats HS but has a safety profile including one or more of the following characteristics:
no clinically relevant increase of risk of infection,
no clinically relevant increase of major bleeding,
no clinically relevant elevation of liver enzymes; allowing for such long treatment.

### Combination:

The pharmaceutical compositions for use in the disclosed methods may also contain additional therapeutic agents for treatment of the particular targeted disorder. For example, a pharmaceutical composition may also include anti-inflammatory agents. Such additional factors and/or agents may be included in the pharmaceutical composition to produce a synergistic effect with LOU064 described herein.

Various therapies may be beneficially combined with LOU064 during treatment of HS.

Therefore, LOU064 may be administered either simultaneously with, or before or after, one or more other therapeutic agent. LOU064 for use in the method of the invention may be administered separately, by the same or different route of administration, or together in the same pharmaceutical composition as the other agents.

In one embodiment, the invention pertains to the method of treating or preventing HS in a subject, comprising administering to the subject, a therapeutically effective dose of LOU064 and at least one other therapeutic agent as a combined preparation for simultaneous, separate or sequential use in therapy. Optionally, the pharmaceutical composition for use in the method of the invention may comprise a pharmaceutically acceptable excipient, as described above.

Products provided as a combined preparation for use in the method of the invention, include a composition comprising LOU064 and the other therapeutic agent(s) together in the same pharmaceutical composition, or LOU064 and the other therapeutic agent(s) in separate form, e.g. in the form of a kit.

In one embodiment, the invention provides a kit for use in the method of the invention, comprising two or more separate pharmaceutical compositions, at least one of which contains LOU064 or a pharmaceutically acceptable salt thereof. In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, LOU064 and the other therapeutic agent may be manufactured and/or formulated by the same or different manufacturers. Moreover, LOU064 and the other therapeutic may be brought together into a combination therapy: (i) prior to release of the combination product to physicians (e.g. in the case of a kit comprising the compound for use in the method of the invention and the other therapeutic agent); (ii) by the physician themselves (or under the guidance of the physician) shortly before administration; (iii) in the patient themselves, e.g. during sequential administration of LOU064 and the other therapeutic agent.

Accordingly, the invention provides the use of LOU064 or a pharmaceutically acceptable salt thereof, for treating or preventing HS, wherein the medicament is prepared for administration with another therapeutic agent. The invention also provides the use of another therapeutic agent for treating or preventing HS, wherein the medicament is administered LOU064 or a pharmaceutically acceptable salt thereof.

The invention also provides LOU064 or a pharmaceutically acceptable salt thereof, for use in a method of treating or preventing HS, wherein said compound is prepared for administration with another therapeutic agent. The invention also provides another therapeutic agent for use in a method of treating or preventing HS, wherein the other therapeutic agent is prepared for administration with LOU064 or a pharmaceutically acceptable salt thereof.

The invention also provides LOU064 or a pharmaceutically acceptable salt thereof, for use in a method of treating or preventing HS, wherein said compound is administered with another therapeutic agent. The invention also provides another therapeutic agent for use in a method of treating or preventing HS, wherein the other therapeutic agent is administered with LOU064 or a pharmaceutically acceptable salt thereof.

The invention also provides the use of LOU064 or a pharmaceutically acceptable salt thereof, for treating and/or preventing HS in a patient in need of such treatment and/or prevention, wherein the patient has previously (e.g. within 24 hours) been treated with another therapeutic agent. The invention also provides the use of another therapeutic agent for treating or preventing HS in a patient in need thereof, wherein the patient has previously (e.g. within 24 hours) been treated with LOU064 or a pharmaceutically acceptable salt thereof.

Such combined therapies include topical treatments (creams [non-steroidal or steroidal], washes, antiseptics,), systemic treatments (e.g., with biologicals, antibiotics, or chemical entities), antiseptics, photodynamic therapy, and surgical intervention (laser, draining or incision, excision).

Examples of oral antibiotics are tetracyclines and clindamycin and rifampicin.

Non-limiting examples of topical HS agents for use LOU064, include benzoyl peroxide, topical steroid creams, topical antibiotics in the aminoglycoside group, such as clindamycin, gentamicin, and erythromycin, resorcinol cream, iodine scrubs, and chlorhexidine.

Non-limiting examples of HS agents used in systemic treatment for use with LOU064, include IL-17 antagonists (ixekizumab, brodalumab, secukinumab CJM112), but as well IL17A/F antagonists such as bimekizumab or IL17C antagonists such as MOR106, tumor necrosis factor-alpha (TNF-alpha) blockers (such as Enbrel® (etanercept), Humira® (adalimumab), Remicade® (infliximab) and Simponi® (golimumab)), interleukin 12/23 blockers (such as Stelara® (ustekinumab), tasocitinib, and briakinumab), IL-23 blockers (such as guselkumab, tildrakizumab and risankizumab) p19 inhibitors, PDE4 inhibitors such as apremilast or Otezla®, or others such as roflumilast), complement pathway inhibitors, such as Factor B inhibitors (for example compounds disclosed in WO2015/009616, or LNPO23 which is also known as 4-((2S,4S)-4-ethoxy-1-((5-methoxy-7-methyl)-1H-indol-4-yl)methyl)piperidin-2-yl)benzoic acid), C5a inhibitors (such as IFX-001 or vilobelimab or CCX168 also known as Avacopan, or BDB001), IL-1 antagonists (canakinumab, gevokizumab, rilonacept, anakinra, or bermekimab), or a bispecific antibodies such as MAS825 or Lutikizumab (IL-1b and IL-1a), Inflammasome inhibitors such as NLRP3 and NLRP5 inhibitors, CXCR1/2 inhibitors, IL-18 antagonists, IL-6 antagonists, IL-36 antagonists, CD20 antagonists, CTLA4 antagonists, IL-8 antagonists, B-cell depletors (particularly CD20 antagonists, such as rituximab, as well as BAFF-R such as ianalumab, and CD40 antagonists such as Iscalimab (CFZ533)), IL-21 antagonists, IL-22 antagonist, IL-36 or IL-36R antagonist, GCSF-inhibitors, VEGF antagonists, CXCL antagonists, MK-2 inhibitors such as zunsemetinib, IRAK4 inhibitors or IRAK4 degraders (such as SAR44656), LTA4H inhibitors, S1P inhibitors, BTK inhibitors, SYK inhibitors such as fostamatinib, MMP antagonists, and defensin antagonists (e.g., receptor decoys, antagonistic antibodies, etc.), as well as broad spectrum oral JAK inhibitors (pan-JAKi) or more specific TYK2 or JAK 1, JAK2 or JAK 3 inhibitors (more specific/selective JAK/TYK inhibitors include for example allosteric inhibitors such as deucrayacitinib or catalytic inhibitors).

Additional HS agents for use in combination with LOU064, during treatment of HS include retinoids, such as Acitretin (e.g., Soriatane®) and isotretinoin, immune system suppressants (e.g., rapamycin, T-cell blockers [e.g., Amevive® (alefacept) and Raptiva® [efalizumab]) cyclosporine, methotrexate, mycophenolate mofetil, mycophenolic acid, leflunomide, tacrolimus, etc.), hydroxyurea (e.g., Hydrea®), sulfasalazine, 6-thioguanine, fumarates (e.g, dimethylfumarate and fumaric acid esters), azathioprine, colchicine, alitretinoin, steroids, corticosteroids, certolizumab, mometasone, rosiglitazone, pioglitazone, botulinum toxin, triamcinolone, IFX-1 (InflaRx), LY-3041658 (Eli Lilly), TE-2232 (Immunwork), NSAIDs, COX inhibitors, prescription narcotics, ketoprofen, codeine, gabapentin, pregabalin gentanyl, antibiotics (topical, oral, IV) (e.g., clindamycin, rifampin, tetracycline, sarecycline, doxycycline, minocycline, lymecycline, trimethoprim-sulfamethoxazole, erythromycin, ceftriaxone, moxifloxacin, metronidazole, separately or as combinations), corticosteroid (injectable or oral), antiandrogen/hormonal therapy (oral contraceptives, spironolactone, finasteride, dutasteride, progesterone IUD, cyproterone acetate, ethinyloestradiol, gestodene, norgestimate, desogestrel, drospirenone, spironolactone), Triamcinolone Acetonide, MED18968, hydroxychloroquine, dapsone, metformin, adapalene, azelaic acid and zinc.

Preferred combinations for used in the disclosed kits, methods, and uses include the PDE4i and JAKi, as well as antibiotics (all oral).

Examples of JAK inhibitors for use in combination are BMS986165, INCB054707, Ruxolitinib, Abrocitinib, Tofacitinib and Baricitinib. Other examples of JAK inhibitors are compounds disclosed in WO2017/089985, WO2018/055550 and WO2018/055551.

A skilled artisan will be able to discern the appropriate dosages of the above HS agents for co-delivery with LOU064.

It is anticipated that LOU064 oral drug exposure could be increased several fold when administered with CYP3A inhibitors, especially strong CYP3A inhibitors, e.g. strong CYP3A4 inhibitors. Likewise, strong inducers of CYP4A, e.g. of CYP3A4, may significantly decrease the exposure and lead to reduced efficacy of LOU064. These properties of LOU064 are not only relevant for the treatment of HS but also for any BTK-mediated condition. Strong CYP3A inhibitors or CYP3A inducers are defined according to the FDA 2020 guidelines. Therefore, a strong CYP3A inhibitors (e.g. CYP3A4 inhibitors) are inhibitors which upon co-administration with LOU064 leads to an increase of the area under the curve (AUC) of more than 5 fold or a decrease of more than 80% in clearance as compared to administration of LOU064 alone. Strong CYP3A inducers (e.g. strong CYP3A4 inducers) are inducers which upon co-administration with LOU064 decrease the AUC by 80% or more (e.g. by 85%, by 90%, by 95%) as compared to administration of LOU064 alone.

Concomitant administration with strong CYP3A inhibitors and/or inducers, e.g. strong CYP3A4 inhibitors and/or inducers may possibly cause substantial changes in LOU064 drug exposure, and should be avoided. Strong CYP3A4 inhibitors include drugs selected from boceprevir, clarithromycin, cobicistat, conivaptan, danoprevir/ritonavir, darunavir/ritonavir, elvitegravir/ritonavir, idelalisib, indinavir, indinavir/ritonavir, itraconazole, ketoconazole, LCL161, lopinavir/ritonavir, mibefradil, nefazodone, nelfinavir, posaconazole, ritonavir, saquinavir, saquinavir/ritonavir, telaprevir, telithromycin, tipranavir/ritonavir, troleandomycin, Viekira pack or/and voriconazole. CYP3A4 inhibitors can also be contained in grapefruit juice.

Therefore, in another preferred embodiment LOU064 is not administered concomitantly with a strong inhibitor and/or inducer of CYP3A4 e.g. as defined hereinabove.

It has further been found that LOU064 can be co-administered with oral contraceptives such as ethinylestradiol or levonorgestrel without a major impact on their exposure and efficacy. Therefore, in a preferred embodiment, LOU064 is co-administered with oral contraceptives.

As a covalent irreversible BTK inhibitor LOU064 acts via irreversible inhibition of BTK which is countered by de novo protein synthesis. Thus, without wishing to be bound by any theory, it is believed that, whereas reconstitution of the B cell pool after B cell depletion can take several months, the restoration of B cell function after BTK inhibition can be achieved shortly after discontinuation, in particular within days. Therefore, if need be, this therapy could rapidly be stopped which provides clinicians and patients with easier and faster reaction capacity when unforeseen circumstances arise.

Especially in light of the COVID-19 pandemic, B cell-depleted patients have a higher risk of infection. Furthermore, the absence of a fully functional adaptive immune response likely leads to a more severe course.

However, since LOU064 does not result in depletion of the pool of B cells, cessation of the therapy leads to a quick restoration of complete B cell function. This gives patients and treating physicians the possibility to quickly respond to infectious disease or vaccination requirements, in particular vaccination with live vaccines and attenuated vaccines.

According to the invention, LOU064 can be administered during an infection, e.g. during a COVID-19 infection. Thus, LOU064 administration can be continued during an infection, e.g. during a COVID-19 infection.

Preferably, LOU064 administration is delayed in patients with an active infection, e.g. COVID-19, until the infection is resolved.

Thus, one embodiment of the invention relates to LOU064 for use in the treatment of HS wherein a patient acutely or previously infected by COVID-19 is treated.

In a further embodiment LOU064 treatment is continued during COVID-19 infection.

In a preferred embodiment LOU064 treatment is interrupted during COVID-19 infection and continued after overcoming the infection.

A still further embodiment of the invention relates to LOU064 for use in the treatment of HS, wherein the patient is vaccinated during LOU064 therapy. Alternatively, the patient can be vaccinated during LOU064 therapy with non-live vaccines. In one embodiment, the patient is vaccinated with quadrivalent Influenza vaccine, the PPV-23 vaccine or the KLH neoantigen vaccine during LOU064 therapy (e.g. at day 15 after initiating LOU064 therapy). In one aspect of this embodiment, the patient receiving quadrivalent Influenza vaccine, achieves a response as defined by a >4-fold increase of anti-hemaglutinin antibody titers at 28 days after vaccination compared to baseline. In another aspect of this embodiment, the patient receiving the PPV-23 vaccine achieves a >2-fold increase of IgG titers 28 days after vaccination compared to baseline. In yet another embodiment, the patient receiving the KLH neoantigen vaccine achieves a T-cell dependent antibody response as measured by anti-KLH IgG and IgM titers 28 days after vaccination.

Another embodiment of the invention relates to LOU064 for use in the treatment of HS, wherein LOU064 treatment is discontinued for vaccination, in particular wherein LOU064 treatment is discontinued 5-10 days, e.g. 7 or 8 days, preferably 6 weeks prior to vaccination and continued after vaccination, e.g. 5-20 days, preferably 5-10 days or most preferably 10-15 days after vaccination. In an alternative embodiment the vaccination is a vaccination with live vaccines and/or attenuated vaccines. In a particular aspect of this embodiment, the patient is vaccinated with quadrivalent Influenza vaccine, the PPV-23 vaccine or the KLH neoantigen vaccine after discontinuing LOU064 treatment (e.g. 5-10 days or 7 or 8 days after discontinuing LOU064 treatment). In one aspect of this embodiment, the patient receiving quadrivalent Influenza vaccine, achieves a response as defined by a >4-fold increase of anti-hemaglutinin antibody titers at 28 days after vaccination compared to baseline. In another aspect of this embodiment, the patient receiving the PPV-23 vaccine achieves a >2-fold increase of IgG titers 28 days after vaccination compared to baseline. In yet another embodiment, the patient receiving the KLH neoantigen vaccine achieves a T-cell dependent antibody response as measured by anti-KLH IgG and IgM titers 28 days after vaccination. LOU064 treatment is then continued starting on Day 29 after vaccination.

LOU064 or a pharmaceutically acceptable salt thereof, is conveniently administered orally. The duration of the oral therapy using a pharmaceutical composition of the present disclosure will vary, depending on the severity of the disease being treated and the condition and personal response of each individual patient. The health care provider will decide on the appropriate duration of oral therapy and the timing of administration of the therapy, using the pharmaceutical composition of the present disclosure. In some embodiments, the patient is treated for HS according to the claimed methods for at least 16 weeks, at least 24 weeks, at least 36 weeks, at least 48 weeks, at least 52 weeks. In some embodiments, the patient is treated for HS in a chronic use.

In one embodiment, the pharmaceutical composition of the present invention for use in the prevention or the treatment of HS, can be in unit dosage of from about 25 mg to about 100 mg of active ingredient(s) for a subject of about 50-70 kg. The therapeutically effective dosage of a compound, the pharmaceutical composition is dependent on the species of the subject, the body weight, age and individual condition, the severity of the contrast-induced nephropathy disorder. A physician, clinician or veterinarian of ordinary skill can readily determine the effective amount of each of the active ingredients necessary to prevent, treat or inhibit the progress of the disorder or disease.

Preferred formulation is a capsule or a tablet composition comprising from about 25 mg to about 100 mg of LOU064, and one or more excipients independently selected from fillers, disintegrants, binders, and optionally lubricant and glidant. In a preferred embodiment, the capsule or tablet composition comprises from about 25 mg of LOU064 or a pharmaceutically acceptable salt thereof, and and one or more excipients independently selected from fillers, disintegrants, binders, and optionally lubricant and glidant. In yet another embodiment, the capsule or tablet composition comprises about 50 mg of LOU064 or about 100 mg of LOU064 and one or more excipients independently selected from fillers, disintegrants, binders, and optionally lubricant and glidant.

Disclosed herein are methods of treating hidradenitis suppurativa (HS), comprising orally administering to a patient in need thereof a dose of about 50 mg to about 200 mg of LOU064, or a pharmaceutically acceptable salt thereof), daily. Said doses can be administered to the patient either with a once a day dosing regimen or twice a day dosing regimen. In another embodiment, the method comprises orally administering to a patient in need thereof a dose of about 25 mg to about 100 mg of LOU064 twice a day (BID). In a preferred aspect of this embodiment, the method comprises orally administering a dose of about 25 mg of LOU064 to said patient twice a day (BID). In another preferred aspect of this embodiment, the method comprises orally administering a dose of about 100 mg of LOU064 to said patient twice a day (BID).

In preferred embodiments of the disclosed methods, uses and kits, the patient achieves a sustained response after one year of treatment, as measured by (simplified) Hidradenitis Suppurativa Clinical Response (HiSCR), HiSCR50, HiSCR75, HiSCR90, Numerical Rating Scale (NRS), Hidradenitis Suppurativa-Physician Global Assessment (HS-PGA), or Dermatology Life Quality Index (DLQI), severity assessment of HS (SASH), or international HS severity score system (IHS4).

In preferred embodiments of the disclosed methods, uses and kits, prior to treatment with LOU064 as disclosed herein, the patient has been previously treated with a systemic agent for HS. In preferred embodiments of the disclosed methods, uses and kits, the systemic agent is selected from the group consisting of a topical treatment, an antibiotic, an immune system suppressant, a TNF-alpha inhibitor, an IL-1 antagonist, and combinations thereof.

In some embodiments of the disclosed methods, uses and kits, prior to treatment with LOU064 as described herein, the patient has not been previously treated with a systemic agent or a topical treatment for HS (i.e. patient is naïve or biological-naïve).

In one embodiments of the disclosed methods, uses and kits, the LOU064 as described herein (or a pharmaceutically acceptable salt thereof), is administered in combination with at least one of an antibiotic, a JAK inhibitor, a TYK2 inhibitor a PDE4 inhibitor or an immunosuppressant.

In preferred embodiments of the disclosed methods, uses and kits, the dose of the LOU064 as described herein is from about 25 mg to about 100 mg BID. In other preferred embodiments of the disclosed methods, uses and kits, the dose of LOU064 is about 25 mg BID or 100 mg BID.

In preferred embodiments of the disclosed methods, uses and kits, the patient has moderate to severe HS.

As used herein, the phrase “moderate to severe” refers to HS disease in which patients have ≥3 active, inflammatory lesions [i.e., deep inflammatory lesions such as abscesses and/or inflammatory nodules], no more than 15 fistulae and at least two anatomical areas need to be involved in HS lesions.

In preferred embodiments of the disclosed methods, uses and kits, the patient is an adult. In some embodiments of the disclosed methods, uses and kits, the HS patient is an adult with moderate to severe HS disease.

In some embodiments of the disclosed methods, uses and kits, the patient is an adolescent patient (≥12 years of age). In some embodiments, the patient is an adolescent patient having moderate to severe HS.

In some embodiments of the disclosed methods, uses and kits, the patient has been diagnosed with HS for at least one year.

In some embodiments of the disclosed methods, uses and kits, the patient does not have extensive scarring as a result of HS (i.e., <20 fistulas, draining or not draining, preferably no more than 15 fistulas).

In some embodiments of the disclosed methods, uses and kits, the patient previously had an inadequate response to conventional systemic HS therapy.

In preferred embodiments of the disclosed methods, uses and kits, prior to treatment with LOU064 the patient has an HS-PGA score of ≥3.

In preferred embodiments of the disclosed methods, uses and kits, the patient achieves a (simplified) HiSCR by week 16 of treatment.

In preferred embodiments of the disclosed methods, uses and kits, the patient achieves a NRS30 (e.g. pain NRS30 or skin itch NRS30) by week 16 of treatment.

In preferred embodiments of the disclosed methods, uses and kits, the patient has a reduction in HS flares by week 16 of treatment.

In preferred embodiments of the disclosed methods, uses and kits, the patient achieves a reduction of ≤6 as measured by the DLQI by week 16 of treatment.

In preferred embodiments, when the disclosed methods, uses or kits are used to treat a population of patients with moderate to severe HS, at least 40% of said patients achieve a HiSCR (sHiSCR, HiSCR50, HiSCR75 or HiSCR90) by week 16 of treatment in response to said administering step.

In another preferred embodiment, when the disclosed methods, uses or kits are used to treat a population of patients with moderate to severe HS, the difference between the responder rate (e.g. patient achieving a HiSCR response (sHiSCR, HiSCR50, HiSCR75 or HiSCR90 response) to the HS treatment) and the responder rate in the placebo treated patients, by week 16 of treatment is at least 15%, at least 25%, or at least 30%.

In preferred embodiments of the disclosed methods, uses and kits, the patient has a reduction in modified Sartorius score by 16 weeks of treatment.

In preferred embodiments of the disclosed methods, uses and kits, the patient has an improvement in DLQI by 16 weeks of treatment.

In preferred embodiments, when the disclosed methods, uses or kits are used to treat a population of patients with moderate to severe HS, at least 25% (and preferably at least 30%) of said patients achieve an NRS30 response by week 16 of treatment in response to said administering step.

In preferred embodiments, when the disclosed methods, uses or kits are used to treat a population of patients with moderate to severe HS, less than 15% of said patients experience an HS flare during 16 weeks of treatment in response to said administering step.

In preferred embodiments of the disclosed methods, uses and kits, the patient is additionally treated with at least one topical medication and at least one antiseptic in combination with LOU064 as described herein.

In preferred embodiments of the disclosed methods, uses and kits, the patient is treated with LOU064 as described herein for at least 16 weeks, at least 24 weeks, at least 36 weeks, at least 48 weeks or at least 52 weeks. Most preferably, the patient is treated for at least 16 weeks.

In preferred embodiments of the disclosed methods, uses and kits, the patient has a reduction in pain, as measured by VAS or NRS, preferably NRS, as early as one or two weeks after the first dose of LOU064.

In preferred embodiments of the disclosed methods, uses and kits, the patient has a reduction in skin itch, as measured by NRS, as early as one or two weeks after the first dose of LOU064. For example, the patient achieves a skin itch NRS30 after 1 or 2 weeks after the first dose of LOU064.

In preferred embodiments of the disclosed methods, uses and kits, the patient has a reduction in CRP (about 25 to about 30% reduction), as measured using a standard CRP assay, as early as one or two weeks after the first dose of LOU064.

In preferred embodiments of the disclosure, LOU064 is in the anhydrous crystalline form A of the free base as disclosed in WO2020/234779 (Example 1). In one aspect of this embodiment, LOU064 form A is in a substantially pure phase.

### General

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural references unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference. The following Examples are presented in order to more fully illustrate the preferred embodiments of the disclosure. These examples should in no way be construed as limiting the scope of the disclosed subject matter, as defined by the appended claims.

### EXAMPLES

### Abbreviation

AE adverse effect
b.i.d. or BID twice daily
q.d. once daily
ECG electrocardiogram
PK Pharmacokinetics
PD Pharmacodynamics
p.o. per os (by mouth=oral)

### Example 1: LOU064 Signature (Gene Downregulated by LOU064) in Transcriptomic Dataset of Hidradenitis Suppurativa

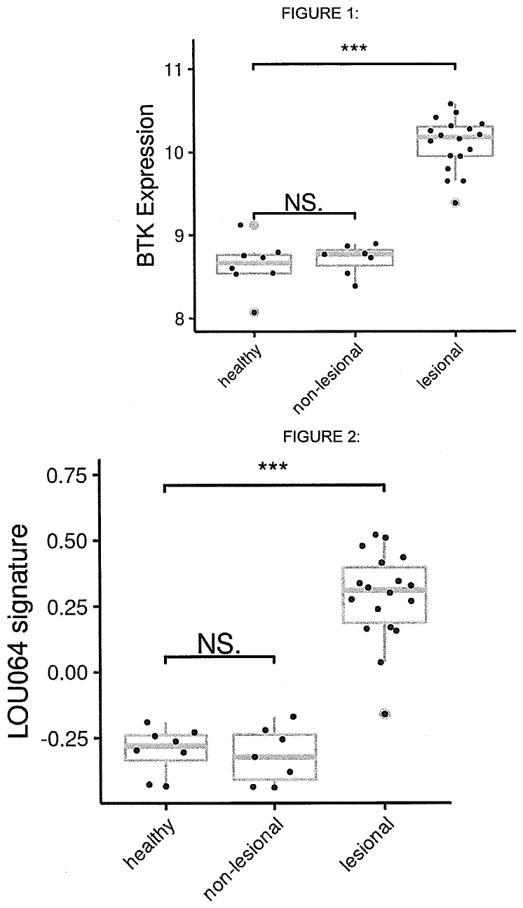

FIG. 1: Gene expression was measured using an Affymetrix GeneChip HG-U133 Plus 2 in skin from healthy donors as well as lesional and non-lesional skin from HS patients (GSE148027). We found BTK expression (measured by probe 205504_at) significantly up-regulated in lesional (but not non-lesional) skin as compared to healthy controls.

FIG. 2 represents LOU signature (LOU064 suppressed genes) in the (bulk) transcriptomic dataset of hidradenitis suppurativa. LOU064 signature was generated by stimulating whole blood with IgM and followed by a treatment with LOU064. Transcriptomic profiles of these samples were measured using Ampliseq. 52 genes that we found significantly down-regulated (FDR<0.05) by LOU064 (as compared to stimulation only) were defined as the “LOU signature”.

The bulk transcriptomics data was measured by Affy chips (Carlos A. Penno et al., J. Invest. Dermatol. 2020, Vol 140, Issue 21, 2421-2432.e10)—49 out of the 52 signature genes were also present in this data and targeted by a total of 106 probes (each gene might be targeted by multiple probes). These 106 probes were used for signature enrichment by an algorithm called GSVA (Sonja Hanzelmann et al. BMC Bioinformatics 2013, 14(7)—https://doi.org/10.1186/1471-2105-14-7) which calculates the enrichment of these 106 probes compared to all other measured probes for each sample (general method on gene set enrichment: Aravind Subramanian et al. PNAS 2005, 102(43) 15545-15550). The results are visualized in FIG. 1. A Wilcoxon test was used to show significantly different signature enrichment between healthy and lesional HS samples (p˜2.7e-7).

The data demonstrate up-regulation of LOU064-suppressed genes (LOU064 signature) in hidradenitis lesions.

### Example 2: Dose Rationale

Translational PK/PD model simulations described below, were used to predict human peripheral tissue occupancy (e.g. spleen and lymph node) of remibrutinib assuming absence of relevant inter-species differences in turn-over and drug-potency. The PK/PD model was focused on B cells, which are, as described above, reported to be the relevant primary target of BTK inhibition in HS. B cells are typically residing in lymph nodes and in the spleen, besides a circulating fraction. In HS, B cells are also present in so called ‘tertiary lymphoid organs’ in HS lesional tissue, resembling key lymph node and germinal center characteristics. B cells residing in spleen, lymph nodes or tertiary lymphoid organs in HS lesions are believed to show a faster BTK turn-over compared to circulating B cells in peripheral blood. The translational PK/PD model simulation guided dose selection for the treatment of HS with LOU064.

### Prediction of BTK Occupancy Using a Translational PKIPD Model for LOU064

BTK occupancy in blood is not an informative biomarker for the purpose of dose selection due to LOU064 pharmacological properties (irreversible binding). It reaches full occupancy even at low doses before showing pharmacological activity through other biomarkers (CD63, CD203c, skin-prick test). Occupancy in tissue may be more representative of the expected efficacy of LOU064.

### Objectives

The purpose of this analysis was to characterize the pharmacokinetics (PK) of LOU064 in healthy volunteers and to use a previously developed, translational target occupancy model to simulate BTK occupancy in human spleen/tissues across a range of doses and dosing regimen (B.I.D vs QD).

### Data

Pharmacokinetic data from a Phase I clinical study reported by Kaul et al. (2021) were used in the current analysis, including 102 patients.

### Methods

A translational target occupancy model to simulate BTK occupancy in spleen/tissues was developed using a two-step approach.

In a first step, a population PK model was established to describe LOU064 PK data from Phase I clinical study reported by Kaul et al. (2021). In a second step, the parameter estimates from the population PK model were used in the BTK occupancy model to predict BTK occupancy in blood and spleen/tissues. Ultimately the BTK occupancy model was used to predict the BTK occupancy in spleen/tissues for different dosing regimens (QD, B.I.D) at different doses.

### Results

A population PK model has been developed to describe the interim PK from a Phase I clinical study reported by Kaul et al. (2021). In order to address the change in clearance after repeated dosing for doses lower than 50 mg (lower clearance at steady state at Day 12 when compared to Day 1 with no difference at higher doses), the clearance was modeled as a function of exponential time decay for doses less than 50 mg and a constant clearance for doses above 50 mg. Overall the resulting population model described the PK data reasonably well.

The PK parameter estimates were used in a translational BTK occupancy model to simulate BTK occupancy at steady state. The BTK occupancy simulations showed that B.I.D dosing is more effective than QD dosing at the same dose to achieve higher BTK occupancy (at trough or averaged over 24-hour interval).

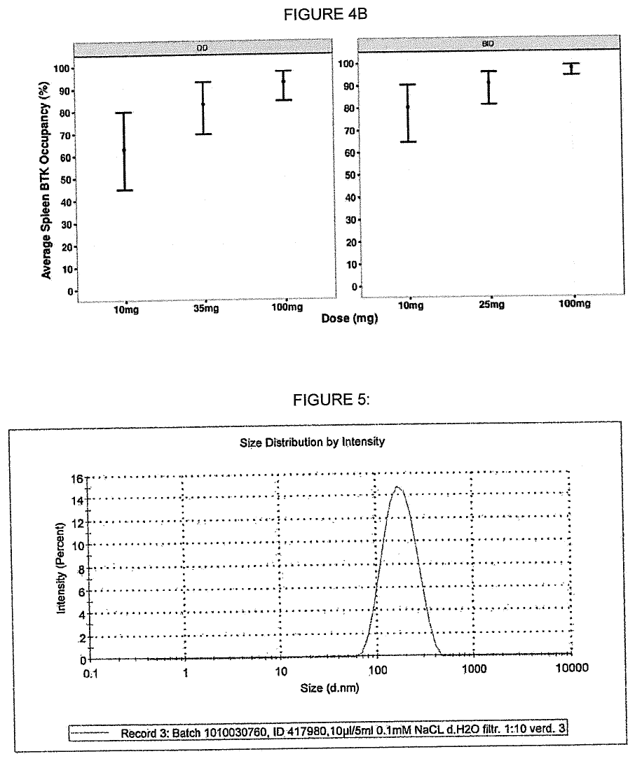

For a selected number of doses at QD and B.I.D regimen, steady-state BTK occupancy at trough and averaged over a period of 24 hours are shown in FIG. 4A (Trough over 24 hours of BTK Occupancy at steady state) and FIG. 4B (Average over 24 hours of BTK Occupancy at steady state), respectively for dosing regimens of 10 mg, 35 mg, 100 mg once daily and 10 mg, 25 mg and 100 mg twice daily. Both figures show that a daily dose up to 200 mg (100 mg B.I.D) may be required to achieve a trough BTK occupancy 80% in a peripheral target tissue.

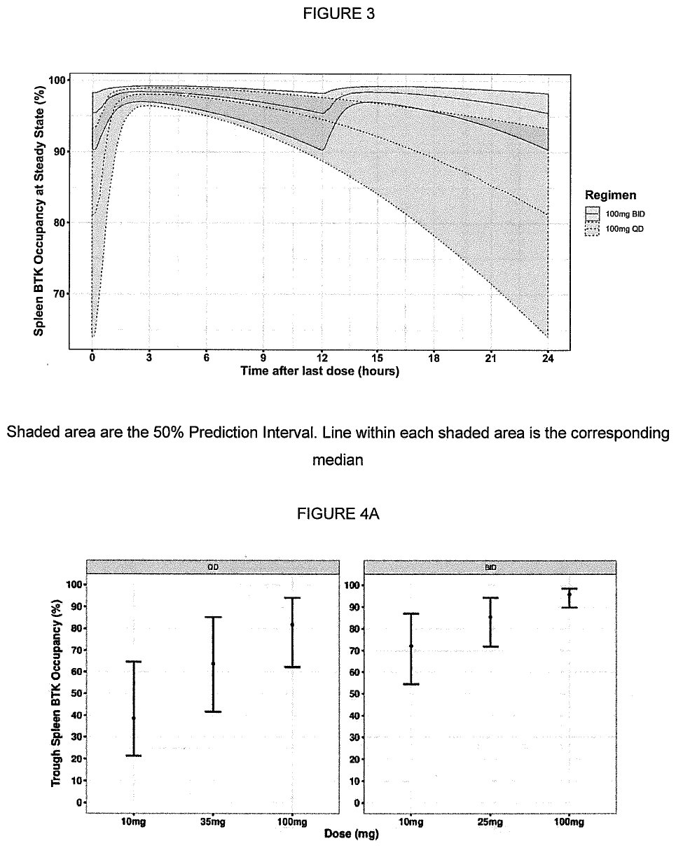

Simulations were performed to compare different dosing regimens. A comparison of simulated spleen BTK occupancy at steady state of 100 mg B.I.D vs. 100 mg QD over time is shown in FIG. 3. The graph shows that the occupancy from B.I.D dosing is higher and less variable compared to QD dosing, as expected from basic principles.

### Conclusions:

The model predicted that overall significantly higher BTK occupancy can be achieved using a b.i.d dosing regimen. While a trough BTK occupancy of approximately 70-95% is predicted in human tissue for the 25 mg b.i.d. dosing regimen, the 100 mg b.i.d. dosing regimen is predicted to achieve ≥90% trough BTK occupancy.

### Example 3: Clinical Trial Design

Blinding of subjects and investigators allows for an unbiased assessment of subjective readouts such as lesion counts in HS or global HS-PGA scores, as well as adverse events.

A randomized, subject and investigator blinded, placebo-controlled, multi-center and parallel-group study is run to assess efficacy, safety and tolerability of several active treatment compounds, such as LOU064 (remibrutinib), in subjects with moderate to severe hidradenitis suppurativa (HS). After a screening period, the treatment period is for 16 weeks and is followed by a safety follow-up of approximately 4 weeks. Subjects are given 25 mg BID p.o. of LOU064, 100 mg BID p.o., or placebo BID p.o. Approximately 70 subjects are randomized, 60 on active (30 will receive 100 mg bid, 30 receive 25 mg bid) and 10 on placebo.

The primary objective is to show preliminary efficacy of treatment with LOU064, in HS subjects after 16 weeks of treatment in comparison to placebo. After the 16-weeks treatment period a follow up period for 4 weeks is included to observe a sustainability of the effect can be sustained or increased after 16 weeks of treatment.

### Protocol Summary

Primary objective(s): The primary objective of this study is to assess the efficacy of the remibrutinib when compared to placebo, in moderate to severe inflammatory HS Patients by comparing the proportion of Patients achieving clinical response defined by the simplified Hidradenitis Suppurativa Clinical Response (HiSCR) after 16 weeks of treatment.

Secondary objectives: To assess the safety and tolerability of remibrutinib in Patients with moderate to severe hidradenitis suppurativa (HS) by (i) number and severity of AEs and (ii) physical examination, vital signs, safety laboratory measurements and ECGs at baseline and repeatedly until study completion visit

Study design: This is a non-confirmatory, randomized, subject and investigator-blinded, placebo-controlled, multi-center and parallel-group study to assess efficacy, safety and tolerability of remibrutinib, in subjects with moderate to severe hidradenitis suppurativa. The maximum duration of any subject's participation in a single cohort may not exceed 25 weeks for remibrutinib and will consist of a 35 day screening period, a 16 week treatment period and will be concluded by a 4 week safety follow. LOU064 (remibrutinib), 100 mg b.i.d. p.o or 25 mg b.i.d. p.o or placebo from Day 1 (Week 1) to Day 113 included (Week 17). Subjects will be randomized to either the remibrutinib treatment arms or its corresponding placebo in a 3:3:1 ratio.

Population: Adult male and female participants, 18 to 65 years of age, presenting with moderate to severe hidradenitis suppurativa, diagnosed with recurrent inflammatory lesions for at least 12 months prior to screening.

Key inclusion criteria: Male and female subjects, 18 to 65 years of age (inclusive), with clinically diagnosed HS for at least 12 months prior to screening; minimal body weight of 50 kg (inclusive) at screening; Patients with moderate to severe HS, as per evaluation at screening (pre-dose on day 1):
A total A total of at least 3 inflammatory lesions, i.e., abscesses and/or inflammatory nodules, and
No more than 15 fistulae, and
At least two anatomical areas need to be involved with HS lesions

### Key Exclusion Criteria:

Use of other investigational drugs at the time of screening, or within 30 days or 5 half-lives of randomization, whichever is longer; or longer if required by local regulations;
women physiologically capable of becoming pregnant, unless they are using highly effective methods of contraception during dosing and for a minimum of 2 weeks after the last administration of remibrutinib
significant bleeding risk or coagulation disorders e.g.:
   History of gastrointestinal bleeding, e.g. in association with use of nonsteroidal anti-inflammatory drugs (NSAID), that was clinically relevant:
   Use of anticoagulant medication [for example, warfarin or Novel Oral Anti-Coagulants (NOAC)] within 2 weeks prior to randomization
   International Normalized Ratio (INR) of more than 1.5 at screening
   Use of anti-platelet medication [including dual anti-platelet therapy (e.g. acetylsalicylic acid+clopidogrel)] within two weeks prior to randomization
Note: monotherapy with acetylsalicylic acid (up to 100 mg/day) or clopidogrel is not exclusionary.
   Major surgery within 8 weeks prior to screening or planned surgery the during study treatment period.

### Key Efficacy Assessments:

Simplified and original Hidradenitis Suppurativa clinical response (HiSCR) rate
International Hidradenitis Suppurativa Severity Score System (IHS4)
Hidradenitis suppurativa-Physician Global Assessment (HS-PGA) score and responder rate
HS inflammatory lesion count
Severity Assessment of Hidradenitis Suppurativa (SAHS)

### Key Safety Assessments:

Number and severity of Adverse Events (CTCAE v5.0 grading)
Physical examination, incl. vital signs, safety laboratory measurements, and ECGs

### Other Assessments:

PK
Skin itch NRS (Cohort D)
Patient reported outcomes including Skin Pain Numerical Rating Scale (NRS), Dermatology Life Quality Index (DLQI), Patient's Global Assessment (PGA).

### Objectives

| Objective(s) | Endpoint(s) |
|---|---|
| Primary objective(s) | Endpoint(s) for primary objective(s) |
| To assess the efficacy of the | Proportion of patients achieving clinical |
| remibrutinib, compared to placebo | response evaluated by the simplified |
| in moderate to severe inflammatory | Hidradenitis Suppurativa Clinical Response |
| HS patients | (HiSCR) after 16 weeks of treatment |
| Secondary objective(s) | Endpoint(s) for secondary objective(s) |
| To assess the safety and tolerability | Number and severity of AEs |
| of remibrutinib in patients with | Physical examination, vital signs, safety |
| moderate to severe HS | laboratory measurements, ECGs at baseline |
| | and repeatedly until study completion visit |
| Exploratory objective(s) | Endpoint(s) for exploratory objective(s) |
| To assess the pharmacokinetics of | remibrutinib concentration in whole blood: pre |
| multiple doses of remibrutinib in | and post treatment during the day 29 and day |
| patients with HS | 113 visit. |
| To explore the effect of the | BTK occupancy in whole blood at baseline, |
| remibrutinib versus placebo on | during treatment and follow-up |
| other efficacy measurements over | Hidradenitis Suppurativa - Physician's Global |
| time | Assessment (HS-PGA) responders over time |
| To explore exposure-response | HS lesion count over time and categorized |
| relationship | reduction from baseline of abscesses and |
| To assess the effect of the | nodules by 50, 75, 90 and 100% (AN 50, 75, 90 |
| investigational treatments | and 100) |
| compared to placebo on patient | Severity assessment of hidradenitis suppurativa |
| reported outcomes (PRO) | (SAHS) Score |
| To explore the potential of | International Hidradenitis Suppurativa Score |
| remibrutinib to reduce HS flares | System (IHS4) |
| versus placebo | remibrutinib concentration in whole blood and |
| To assess clinical activity of | HS lesion count |
| remibrutinib in moderate to severe | Dermatology Life Quality Index (DLQI) |
| inflammatory HS patients over time | Patient's Global Assessment (PGA) |
| To explore dose response | Proportion of patients achieving Pain NRS30 |
| relationship of remibrutinib (100 mg | after 16 weeks of treatment, among patients |
| b.i.d and 25 mg b.i.d.) in patients | with baseline Skin Pain NRS ≥ 3 using the |
| with HS | Numerical Rating Scale of Pain Assessment |
| | (NRS) |
| | Proportion of patients achieving itch NRS30 |
| | after 16 weeks of treatment, among patients |
| | with baseline Skin itch NRS ≥ 3 using the |
| | Numerical Rating Scale of itch Assessment |
| | (NRS) |
| | Number of new boils or existing boils which flare |
| | up in the past four weeks |
| | Proportion of patients who experience at least |
| | one flare over 16 weeks of treatment |
| | Proportion of patients achieving clinical |
| | response evaluated by the HiSCR and |
| | simplified HiSCR at each visit |
| | Cohort D only: collect and compare dosing |
| | regimen data over time in HiSCR and HS lesion |
| | counts and others (such as skin pain and itch) |

### Clinical Assessments Additional References:

simplified HiSCR was selected as the primary endpoint. The original HiSCR used in the phase 3 clinical trials conducted with adalimumab (Kimball et al 2016 N Engl J Med 375:422-34) has been validated. The simplified HiSCR is defined as 50% decrease in the total number of abscesses plus inflammatory nodules, without any increase in draining fistulae. However, in contrast to the original HiSCR, increases in abscesses are allowed to constitute a clinical response, if the previous conditions are met.
The inflammatory lesions of HS will be counted as individual lesions (inflammatory nodules, abscesses and draining fistulae) in the typical anatomical areas. In addition to the count, a global assessment scale (Hidradenitis Suppurativa-Physician's Global Assessment or HS-PGA) as well as a composite score (Severity Assessment of Hidradenitis suppurativa score or SAHS) will be used. Abscesses and nodules will also be presented as AN Counts.
HS-PGA (Hidradenitis Suppurativa-Physician Global Assessment): The score will be used as exploratory objective to assess HS and was used and described in Kimball A B, Kerdel F, Adams D, et al (2012) Adalimumab for the treatment of moderate to severe Hidradenitis suppurativa: a parallel randomized trial. Ann Intern Med; 157:846-55.
The SAHS score is a composite score (Hessam S, Scholl L, Sand M, et al (2018) A Novel Severity Assessment Scoring System for Hidradenitis Suppurativa. JAMA Dermatol; 154(3):330-335.) and will be derived from the collected information for inflammatory lesion count, the fistulae count, and the NRS pain. In addition, the anatomical areas and the new or flared existing boils will be collected in both cohorts.
Several patient reported outcomes will be used, including the Dermatology Life Quality Index (DLQI). Finally, as from a subject's perspective skin related pain is the most important symptom, the numerical rating scale (NRS) for pain is included.
Other Patient reported outcomes (PRO) will include Dermatology Life Quality Index (DLQI) as dermatology related Quality of life (QoL) tool with validated scores available in many countries and languages. It includes as well a Patient Global Assessment.
Skin Pain—NRS (numerical rating scale for pain): An NRS for skin related pain was used in adalimumab studies (Kimball et al. (2016) N Engl J Med 375:422-34) and will be used as skin or HS related pain is one of the highest burden for the patient (Matusiak et al (2017) J Am Acad Dermatol; 76:670-5). The pain that is HS related will be recorded on average in the last 24 hours and at worst (in the last 24 hours).
As itch is now recognized as a symptom in HS patients, skin itch NRS has been included (Nguyen et al 2021, Fernandez et al 2021).

### Example 5: 100 mg Film Coated Tablet

Below a preferred pharmaceutical composition (film-coated tablet) is illustrated.

| | Amount per 100 mg | |
|---|---|---|
| | Film-coated | |
| Ingredient | tablet (mg) | Function |
| Tablet core | | |
| LOU064 | 100.0 | Drug substance |
| Mannitol | 243.8 | Carrier |
| Cellulose, microcrystalline/ | 85.8 | Diluent |
| Microcrystalline Cellulose | | |
| Copovidone | 50.0 | Binder |
| Croscarmellose sodium | 31.2 | Disintegrant |
| Sodium Stearyl Fumarate | 5.2 | Lubricant |
| Sodium laurilsulfate/Sodium | 4.0 | Surfactant |
| lauryl sulfate | | |
| Water, purified/Purified | — | Suspending agent/ |
| water¹ | | Solvent |
| Core tablet weight | 520.0 | |
| Coating | | |
| Basic coating premix, yellow | 14.2 | Film-coating |
| Basic coating premix, red | 4.4 | Film-coating |
| Basic coating premix, white | 4.4 | Film-coating |
| Basic coating premix, black | 1.2 | Film-coating |
| Water, purified² | — | Granulation liquid |
| Total film-coated | 544.2 | |
| tablet weight | | |

^{1, 2}Removed during processing

### Example 6: 50 mg Film Coated Tablet

Below a preferred pharmaceutical composition (film-coated tablet) is illustrated.

| | Amount per 50 mg | |
|---|---|---|
| | Film-coated | |
| Ingredient | tablet (mg) | Function |
| Tablet core | | |
| LOU064 | 50.0 | Drug substance |
| Mannitol | 121.9 | Carrier |
| Cellulose, microcrystalline/ | 42.9 | Diluent |
| Microcrystalline Cellulose | | |
| Copovidone | 25.0 | Binder |
| Croscarmellose sodium | 15.6 | Disintegrant |
| Sodium Stearyl Fumarate | 2.6 | Lubricant |
| Sodium laurilsulfate/Sodium | 2 | Surfactant |
| lauryl sulfate | | |
| Water, purified/Purified | — | Suspending agent/ |
| water¹ | | Solvent |
| Core tablet weight | 260.0 | |
| Coating | | |
| Basic coating premix, yellow | 7.2 | Film-coating |
| Basic coating premix, red | 2.2 | Film-coating |
| Basic coating premix, white | 2.2 | Film-coating |
| Basic coating premix, black | 0.6 | Film-coating |
| Water, purified² | — | Granulation liquid |
| Total film-coated | 272.1 | |
| tablet weight | | |

^{1, 2}Removed during processing

### Example 7: 25 mg Film Coated Tablet

Below a preferred pharmaceutical composition (film-coated tablet) is illustrated.

| | Amount per 25 mg | |
|---|---|---|
| | Film-coated | |
| Ingredient | tablet (mg) | Function |
| Tablet core | | |
| LOU064 | 25.0 | Drug substance |
| Mannitol | 60.9 | Carrier |
| Cellulose, microcrystalline/ | 21.5 | Diluent |
| Microcrystalline Cellulose | | |
| Copovidone | 12.5 | Binder |
| Croscarmellose sodium | 7.8 | Disintegrant |
| Sodium Stearyl Fumarate | 1.3 | Lubricant |
| Sodium laurilsulfate/Sodium | 0.9 | Surfactant |
| lauryl sulfate | | |
| Water, purified/Purified | — | Suspending agent/ |
| water¹ | | Solvent |
| Core tablet weight | 130.0 | |
| Coating | | |
| Basic coating premix, yellow | 3.6 | Film-coating |
| Basic coating premix, red | 1.1 | Film-coating |
| Basic coating premix, white | 1.1 | Film-coating |
| Basic coating premix, black | 0.3 | Film-coating |
| Water, purified² | | Granulation liquid |
| Total film-coated tablet | 136.0 | |
| weight | | |

^{1, 2}Removed during processing

### Example 8: Safety of LOU064

The safety of LOU064 has been tested in Phase I and Phase II pharmacokinetic and clinical pharmacology healthy subject studies and in Phase II/Phase Ill clinical studies conducted with patients suffering from indications other than MS, particularly chronic spontaneous urticaria (CSU) and Sjögren's Syndrome (SjS).

### Short-Term Safety of LOU064 in Phase I Clinical Study

Short-term safety of LOU064 as a single dose or as multiple doses for up to 18 days covering the dose range from 0.5 mg to 600 mg for up to 18 days and further at 100 and 200 mg b.i.d. for up to 12 days has been shown in Phase I clinical studies (Kaul, M. et al. (2021). Remibrutinib (LOU064): A selective potent oral BTK inhibitor with promising clinical safety and pharmacodynamics in a randomized phase I trial. Clinical and Translational Science. 10.1111/cts.13005).

### Summary of Safety in Phase 2b Study (Extension Phase) in CSU Subjects (Interim Results)

In a 52-week open label extension study to evaluate the long-term safety and tolerability of LOU064 in eligible subjects with CSU who participated in the Phase 2b study the dose used was 100 mg b.i.d.

No safety signal has been observed based on an interim analysis of the 100 subjects who received at least 1 dose of LOU064 with a median exposure of 17.86 weeks (range: 2.9 weeks to 44.7). At the time of cut-off, 93 subjects (93%) were ongoing, and 7 subjects had discontinued from the study; none of the discontinuations were due to adverse events.

Table 4 presents the safety summary observed in the Phase 2b study up to the cut-off date for the interim analysis.

TABLE 4
Interim analysis for open label extension study: Deaths,
other serious or clinically significant adverse events
or related discontinuations (Safety Set)

| | LOU064 |
|---|---|
| | 100 mg B.I.D. |
| | N = 100 |
| | n (%) |
| Patients with AE(s) | 58 (58) |
| Patients with serious | |
| or other significant | |
| events | |
| Death | 0 |
| Non-fatal SAE | 3 (3.0) |
| Discontinued study due to any AE(s) | 0 |
| Discontinued study due to an SAE(s) | 0 |
| Treatment interruption due to AE(s) | 5 (5.0) |
| Treatment interruption due to SAE(s) | 1 (1.0) |

Interim analysis cut off 31 Aug. 2020

Fifty-eight subjects (58%) experienced at least one treatment emergent AE. The majority of AEs were non-serious, did not lead to treatment discontinuation and were mild in severity. The most frequently affected SOC were Infections and infestations (14%) followed by Skin and subcutaneous tissue disorders (13%) with no trends with respect to specific adverse events. The most common adverse event preferred terms (2%) were headache (6%), diarrhea (4%), dizziness (3%) and gastroenteritis (3%); no bleeding events (defined as events under Haemorrhages SMQ broad and the PTs including Platelet aggregation abnormal, Platelet aggregation decreased, Platelet aggregation inhibition, Platelet dysfunction, Platelet function test abnormal and Platelet toxicity) or events under SOC Blood and lymphatic system disorders were reported. Three SAEs were reported: ovarian cyst, chest pain and appendicitis; none were considered related to study drug.

Conclusions from the Phase 2b Study and Corresponding Open Label Extension Study

Taken together, there have been no safety findings in the Phase 2b study, across all the doses assessed. Furthermore, in the corresponding CSU extension study, which uses LOU064 100 mg b.i.d. open-label, no safety signals have been observed in 100 subjects enrolled as of 31 Aug. 2020. The proposed highest dose of 100 mg LOU064 b.i.d. is considered to be well tolerated and with a favorable safety profile.

Summary of Safety in Phase 2b Study (Extension Phase) in CSU Subjects (Interim Results/Patients with Medium Exposure of 35.14 Weeks)

In the above 52-week open label extension study to evaluate the long-term safety and tolerability of LOU064 in eligible subjects with CSU who participated in the Phase 2b study with a dose of 100 mg b.i.d., a new interim analysis was performed with patients (N=183) with a medium exposure of 35.14 weeks and the results were compared to the safety results in the randomized double-blind, placebo-controlled Ph2b core study, in adult patients with CSU who received (1:1:1:1:1:1:1) remibrutinib 10 mg qd (once daily), 35 mg qd, 100 mg qd, 10 mg bid (twice daily), 25 mg bid, or 100 mg bid or placebo up to 12 weeks (wks) (NCT03926611). (Table 5) In the long-term exposure of the ES (median 35.14 wks, N=183), the proportion of patients with at least one adverse effect (AE) on remibrutinib treatment (57.4% [n=105]) was similar to the CS (presented through for any remibrutinib dose) (58.1% [n=155]; median 12.14 wks, N=267). In the ES, there were 4 serious adverse effects (SAEs), 6 AEs leading to treatment discontinuation and no deaths. The incidences of AEs by primary organ class (SOC) reported in the ES and CS were similar: infections and infestations (23.0% and 24.0%), followed by skin/subcutaneous tissue disorder (17.5% and 16.9%) (Table 5). Incidences of reported AEs by preferred term were comparable in ES and CS with headache (6.6% and 9.7%) being most common. Incidence of AESI in the ES such as infections (23%), bleeding (4.4%) and cytopenias (0.5%) were in line with the CS. Newly occurring notable transaminase increases were single in both ES (isolated ALT>3×ULN, normalized within 4 weeks, in 1 patient discontinued early for personal reasons) and CS (ALT>5×ULN in 1 patient, normalised on treatment). The analysis of laboratory parameters did not reveal significant safety concerns and no clinically meaningful changes in vital signs were observed. There were no significant ECG findings or QT of >500 ms noted in any patient.

TABLE 5
Safety profile of remibrutinib(LOU064) in Phase 2b core and extension study (safety set)

| Core study (CS) | | | | | | | | | ES |
|---|---|---|---|---|---|---|---|---|---|
| | LOU064 | | | | | | | | LOU064 |
| | 10 mg | 35 mg | 100 mg | 10 mg | 25 mg | 100 mg | Any | | 100 mg |
| Patients | q.d. | q.d. | p.b | b.i.d. | b.i.d. | b.i.d. | dose | Placebo | b.i.d. |
| n (%) | N = 44 | N = 44 | N = 47 | N = 44 | N = 43 | N = 45 | N = 267 | N = 42 | (N = 183) |
| Patients with ≥1 | 29 | 23 | 27 | 21 | 26 | 29 | 155 | 18 | 105 |
| AE | (65.9) | (52.3) | (57.4) | (47.7) | (60.5) | (64.4) | (58.1) | (42.9) | (57.4) |
| Discontinued | 0 | 0 | 0 | 3 | 1 | 3 | 7 | 0 | 6 |
| study | (0.0) | (0.0) | (0.0) | (6.8) | (2.3) | (6.7) | (2.6) | (0.0) | (3.3) |
| treatment due | | | | | | | | | |
| to AE(s) | | | | | | | | | |
| Patients with | 1 | 0 | 0 | 2 | 2 | 0 | 5 | 0 | 4 |
| SAE(s) | (2.3) | (0.0) | (0.0) | (4.5) | (4.7) | (0.0) | (1.9) | (0.0) | (2.2) |
| Most frequent AEs by primary system organ class (≥10% of all patients | | | | | | | | | |
| receiving any LOU064 dose or placebo group in CS or in 100 mg b.i.d. in ES) | | | | | | | | | |
| Infections and | 12 | 9 | 14 | 6 | 12 | 11 | 64 | 9 | 42 |
| infestations | (27.3) | (20.5) | (29.8) | (13.6) | (27.9) | (24.4) | (24.0) | (21.4) | (23.0) |
| Skin and | 7 | 9 | 5 | 6 | 12 | 6 | 45 | 2 | 32 |
| subcutaneous | (15.9) | (20.5) | (10.6) | (13.6) | (27.9) | (13.3) | (16.9) | (4.8) | (17.5) |
| tissue | | | | | | | | | |
| disorders | | | | | | | | | |
| Nervous | 3 | 10 | 7 | 4 | 6 | 5 | 35 | 7 | 19 |
| system | (6.8) | (22.7) | (14.9) | (9.1) | (14.0) | (11.1) | (13.1) | (16.7) | (10.4) |
| disorders | | | | | | | | | |
| Gastrointestinal | 7 | 4 | 6 | 6 | 2 | 5 | 30 | 5 | 26 |
| diseases | (15.9) | (9.1) | (12.8) | (13.6) | (4.7) | (11.1) | (11.2) | (11.9) | (14.2) |
| Most frequent AEs by PT (≥5% of patients in dose or placebo group in CS or in | | | | | | | | | |
| 100 mg b.i.d. in ES) | | | | | | | | | |
| Headache | 1 | 7 | 4 | 3 | 6 | 5 | 26 | 6 | 12 |
| | (2.3) | (15.9) | (8.5) | (6.8) | (14.0) | (11.1) | (9.7) | (14.3) | (6.6) |
| Nasopharyngitis | 7 | 2 | 2 | 4 | 4 | 4 | 23 | 3 | 6 |
| | (15.9) | (4.5) | (4.3) | (9.1) | (9.3) | (8.9) | (8.6) | (7.1) | (3.3) |
| Chronic | 3 | 2 | 3 | 4 | 2 | 2 | 16 | 1 | 6 |
| spontaneous | (6.8%) | (4.5%) | (6.4%) | (9.1%) | (4.7%) | (4.4%) | (6.0%) | (2.4%) | (3.3) |
| urticaria | | | | | | | | | |

### CONCLUSION

Remibrutinib showed a favorable safety profile across the whole dose range with no new safety signals observed over longer-term exposure to 100 mg bid dose up to 52 wks in patients with CSU.

### Example 9: Evaluation of the Modulation of Immune Response to Three Different Types of Vaccines by Concomitant and Interrupted Administration of Remibrutinib in Health Subject

### Objectives and Related Endpoints

| Objective(s) | Endpoint(s) |
|---|---|
| Primary objective(s) | Endpoint(s) for primary objective(s) |
| To evaluate non-inferiority of concomitant and | Achievement of response where |
| interrupted remibrutinib treatment on the | response is defined as: |
| immune response following vaccinations in | Influenza: >4-fold increase of |
| healthy participants relative to placebo for a: | anti- hemagglutinin antibody titers |
| T cell-dependent vaccine (Seasonal | at 28 days (Day 43) after |
| Influenza, quadrivalent vaccine) | vaccination compared with |
| T cell-independent vaccine (PPV-23, | baseline (i.e. sero-conversion) |
| Pneumovax ®, Merck & Co. Inc., USA) | PPV-23: >2-fold increase of |
| | immunoglobulin G (IgG) titers 28 |
| | days (Day 43) after vaccination |
| | compared with baseline. |
| Secondary objective(s) | Endpoint(s) for secondary objective(s) |
| To assess the effect of concomitant and | T-cell dependent antibody response |
| interrupted remibrutinib treatment on the | as measured by anti-KLH IgG and |
| immune response following vaccinations in | IgM titers 28 days after vaccination |
| healthy participants relative to placebo for T | (Day 43) |
| cell-dependent de novo vaccine (KLH, | All safety assessments (including vital |
| Immucothel ®) | signs, ECGs, safety laboratory |
| To investigate the safety and tolerability of | parameters, and AEs) |
| remibrutinib administered as 100 mg b.i.d. for | All safety assessments (including vital |
| up to 35 days in healthy participants | signs, ECGs, safety laboratory |
| To explore the safety and tolerability of the | parameters, and AEs) |
| vaccinations administered to healthy | PK parameters: AUCtau (Day 15 |
| participants receiving remibrutinib | only), AUClast, Cmax, Tmax |
| To assess the PK of remibrutinib at a 100 mg | |
| b.i.d. dose | |

### Overall Design

This randomized, double-blind, placebo-controlled study has a parallel group design. Approximately 90 healthy female of non-childbearing potential and male participants are randomized to any of the three treatment groups in order to achieve a minimum of 72 evaluable completers considering an estimated drop-out rate up to 20%. The study will consist of a 28-day screening period, a 43-day treatment period, followed by a Study Completion evaluation (Day 57) within two weeks after last study drug administration. A safety follow-up call is performed approximately 30 days after the last study drug administration (Day 73). Participants are domiciled on Days −1 to 1 and Days 14-17. In total, the maximum study duration for each participant is about 85 days.

The impact of concomitant and interrupted remibrutinib treatment scenarios for Influenza/Pneumovax© 23 and Immucothel® is evaluated with reference to placebo.

### Study Conduct

### Screening & Baseline

Participants who meet the eligibility criteria at screening will be admitted to baseline evaluations on Day −1. All baseline safety evaluation results must be available prior to first dosing. At baseline, participants are randomized to one of the three treatment groups described below.

### Treatment

All participants receive study drug (remibrutinib 100 mg or placebo b.i.d.) from Day 1 until Day 42 and return to the clinic for End of Treatment visit at day 43. All participants also receive the quadrivalent Influenza vaccine, the PPV-23 vaccine and the KLH neoantigen vaccine on Day 15. Vaccinations should occur 3 hours after study drug administration.

During clinical visits and during domiciliation (Days −1 to 1 and Days 14-17), participants are administered the study drug by the study personnel at the clinic. Upon discharge from clinical visits during the Treatment period, study drug is provided to the participants for self-administration at home, along with the medication diary.

Safety assessments will include physical examinations, ECGs, vital signs, standard clinical laboratory evaluations (hematology, blood chemistry, urinalysis) adverse event and serious adverse event monitoring.

Multiple blood samples to assess remibrutinib pharmacokinetics will be drawn from all participants on Day 8, Day 15 and on Day 36.

### Group A (Concomitant Remibrutinib Treatment):

Participants will receive placebo (b.i.d.) from Days 1-7, followed by treatment with remibrutinib (100 mg b.i.d.) on study Days 8-15 to achieve PK/PD steady state, prior to administration of the three vaccines on Day 15. Participants will continue to receive remibrutinib (100 mg b.i.d.) until Day 42.

### Group B (Interrupted Remibrutinib Treatment):

Participants will be treated with remibrutinib 100 mg b.i.d from Day 1-7 to achieve PK/PD steady state conditions, followed by placebo (b.i.d.) administration from Day 8-28 and will be administered the three vaccines on Day 15. Treatment with remibrutinib 100 mg b.i.d. will be re-initiated treatment from Day 29 to 42.

### Group C (Placebo):

Participants in Group C will receive placebo (b.i.d) from Day 1-42 and will be vaccinated with the 3 vaccines on Day 15 under placebo conditions.

### Key INCLUSION Criteria

Signed informed consent must be obtained prior to participation in the study.
Healthy, or mildly obese but otherwise healthy, male and non-childbearing potential female participants aged 18 to 55 years (inclusive).
Participants should be in good health as determined by past medical history, physical examination, vital signs, ECG, and laboratory tests at Screening and Baseline visit as indicated.
At Screening and Baseline, vital signs (systolic and diastolic blood pressure and pulse rate) will be assessed in the sitting position and again (when required by the assessment schedule) in the standing position. Sitting vital signs (after sitting 3 minutes) should be within the following ranges:
   Tympanic body temperature of 35.0 to 37.5° C.
   Systolic blood pressure (SBP) of 90 and 139 mmHg (inclusive).
   Diastolic blood pressure of (DBP) 50 and 89 mmHg (inclusive).
   Pulse rate of 45 and 90 bpm (inclusive).
Participants must weigh at least 50 kg to participate in the study and must have a body mass index (BMI) within the range of 18 to 34.9 kg/m2.
Participants must be willing to remain at the clinical site as required by the protocol and to comply with the requirements/instructions outlined in the ICF.
Able to read, speak, and understand the local language, to understand and comply with the requirements of the study.

Key Exclusion criteria
1. Use of other investigational drugs within 5 half-lives or 30 days prior to first dosing, whichever is longer.
2. Current evidence or past medical history of clinically significant ECG abnormalities or a family history (grandparents, parents, and siblings) of a prolonged QT interval syndrome or other abnormalities in cardiac conduction, history of additional risk factors for Torsade de Pointes (TdP) (e.g. heart failure, hypokalemia) and/or known history or current clinically significant arrhythmias. Abnormal ECG defined as PR>220 msec, QRS complex >120 msec, for males and females QTcF>450 msec, or any other morphological changes, other than early repolarization, nonspecific S-T or T-wave changes.
3. History or presence of malignancy of any organ system (other than localized basal cell carcinoma of the skin or in-situ cervical cancer), treated or untreated, within the past 5 years, regardless of whether there is evidence of local recurrence or metastases.
4. History or presence of any clinically significant disease of any major system organ class including (but not limited to) cardiovascular, pulmonary, metabolic, hepatic, renal, hematologic, endocrine, neurological or psychiatric diseases which had not resolved within two weeks prior to initial dosing
5. Hypersensitivity to remibrutinib or drugs from the same compound class or its excipients.
6. Any contraindication for the use of the Pneumovax 23, influenza or KLH vaccine including any acute infection, fever or hypersensitivity reactions or known hypersensitivity to any relevant component of the vaccines to be administered in this study (e.g., hen's egg or shellfish/KLH).
7. History of vaccination with the 2022-2023 seasonal influenza vaccine or known clinical diagnosis of influenza infection during the 2022-2023 influenza season prior to enrollment.
8. History of previous exposure or immunization with KLH.

1. A method of treating or preventing hidradenitis suppurativa (HS), comprising administering to a patient in need thereof a therapeutically effective dose of a BTK inhibitor, e.g. a selective BTK inhibitor, e.g. LOU064.

2. The method according to claim 1, comprising administering to a patient in need thereof a therapeutically effective dose of LOU064 wherein said dose is from about 50 mg to about 200 mg daily.

3. The method according to claim 2, wherein the therapeutically effective dose of LOU064 is from about 25 mg twice a day to about 100 mg twice a day.

4. The method according to claim 2, wherein the therapeutically effective dose of LOU064 is about 100 mg twice daily.

5. The method according to claim 2, wherein the therapeutically effective dose of LOU064 is about 25 mg twice daily.

6. The method according to claim 2, wherein LOU064 is administered for a short term, e.g. less than 6 months, preferably less than 3 months.

7-11. (canceled)

12. The method according to claim 2, wherein LOU064 or a pharmaceutical composition comprising it, is administered in combination with one or more second therapeutic agents.

13. (canceled)

14. (canceled)

15. (canceled)

16. The method according to claim 2 wherein the patient is selected according to at least one of the following criteria:
a) the patient has moderate to severe HS;
b) prior to treatment with the LOU064, the patient has at least 3 inflammatory lesions; or
c) prior to treatment with the LOU064, the patient does not have extensive scarring no more than 15 fistulae) as a result of HS;
d) the patient has a clinical diagnosis of HS for at least 12 months;
e) the patient has at least two anatomical areas involved with HS lesions.

17. The method according to claim 2, wherein said patient achieves by week 16 of treatment at least one of the following:
a) a simplified HiSCR;
b) a reduction in HS flares;
c) a pain reduction as measured by pain NRS, e.g. a pain NRS30;
d) a skin itch reduction as measured by itch NRS, e.g. a skin itch NRS30
f) a reduction in total or different types of HS inflammatory lesion counts, e.g. AN50, AN75, AN90 or AN100;
g) a reduction in IHS4 score;
h) a reduction of ≤6 as measured by the DLQI; and/or
i) an improvement in DLQI.

18. The method according to claim 2, wherein, when said method is used to treat a population of patients with moderate to severe HS, at least 40% of said patients achieve by week 16 at least one of the following:
a) a HiSCR50;
b) a HiSCR75;
c) a HiSCR90; or
d) a simplified HiSCR.

19. The method according to claim 2, wherein by week 16 of treatment, at least 25% of said patients achieve a NRS30 response (e.g. a pain NRS30 or a skin itch NRS30 response); or less than 15% of said patients experience an HS flare.

20. The method according to claim 2, wherein the patient has at least one of the following as early as one or two weeks after the first dose of LOU064:
a) a reduction in pain, as measured by pain NRS, e.g patient achieving pain NRS30,
b) a reduction in skin itch, as measured by itch NRS; e.g. patient achieving skin itch NRS30,
c) a reduction in CRP, as measured using a standard CRP assay, at least 25% reduction.

21. The method according to claim 2 wherein said patient achieves a sustained response 3 months after the end of the treatment, as measured by inflammatory lesion count e.g. AN50, Hidradenitis Suppurativa Clinical Response (HiSCR) (e.g. sHiSCR, HiSCR50, HiSCR75 or HiSCR90), itch or pain Numerical Rating Scale (NRS), Hidradenitis Suppurativa-Physician Global Assessment (HS-PGA), severity assessment of HS (SASH), international HS severity score system (IHS4) or Dermatology Life Quality Index (DLQI).

22. The method according to claim 21, wherein said patient achieves a sustained response 3 months after the end of treatment, as measured by the simplified HiSCR (sHiSCR), HiSCR50, HiSCR75 or HiSCR90.

23. The method according to claim 2 wherein LOU064 is disposed in a pharmaceutical composition, wherein said pharmaceutical composition comprises one or more pharmaceutically acceptable carriers, each of which is independently selected from a filler, a lubricant, a binder, a desintegrant and a glidant.

24. The method according to claim 23, wherein
the pharmaceutical composition is in tablet or capsule form.

25-31. (canceled)

32. The method according to claim 2 wherein LOU064 is a crystalline form of the anhydrous free base characterized by an x-ray powder diffraction pattern comprising one or more representative peaks in terms of 2θ selected from the group consisting of 7.8±0.2° 2θ, 9.2±0.2 °2θ, 12.0±0.2 °2θ, 13.6±0.2 °2θ, 15.6±0.2 °2θ, 16.0±0.2 °2θ, 17.8±0.2 °2θ, 18.3±0.2 °2θ, 18.7±0.2 °2θ, 19.2±0.2 °2θ, 19.9±0.2 °2θ, 22.1±0.2 °2θ, 23.4±0.2 °2θ, 23.9±0.2 °2θ, 24.8±0.2 °2θ, 25.2±0.2 °2θ, 25.5±0.2 °2θ, 27.2±0.2 °2θ, and 29.6±0.2 °2θ, when measured at a temperature of about 25° C. and an x-ray wavelength, λ, of 1.5405 Å.
